(19) United States
(12) Patent Application Publication
(10) Pub. No.: US 2013/0172244 A1
(43) Pub. Date: Jul. 4, 2013

(54) SUBCUTANEOUS THERAPEUTIC USE OF DPP-4 INHIBITOR

(71) Applicants: Thomas KLEIN, Radolfzell (DE); Michael MARK, Biberach an der Riss (DE)

(72) Inventors: Thomas KLEIN, Radolfzell (DE); Michael MARK, Biberach an der Riss (DE)

(21) Appl. No.: 13/727,667

(22) Filed: Dec. 27, 2012

(30) Foreign Application Priority Data
Dec. 29, 2011 (EP) 11196122.3
May 24, 2012 (EP) 12169267.7

(51) Int. Cl. A61K 31/522; A61K 38/26

(52) U.S. Cl. CPC A61K 31/522; A61K 38/26
USPC 514/5.3; 514/11.7; 514/263.21; 514/7.2

(57) ABSTRACT

The present invention relates to methods for treating and/or preventing metabolic diseases comprising the subcutaneous or transdermal administration of a therapeutically effective amount of a certain DPP-4 inhibitor. The invention further relates to a subcutaneous combination of a certain DPP-4 inhibitor and GLP-1 having a short half life, particularly for reducing weight.

### FIELD OF THE INVENTION

The present invention relates to a method for treating and/or preventing metabolic diseases, especially type 2 diabetes mellitus, obesity, overweight, type 1 diabetes, LADA and/or conditions related thereto (e.g. diabetic complications), said method comprising or consisting essentially of administering a therapeutically effective amount of a certain DPP-4 inhibitor (particularly linagliptin) by subcutaneous or transdermal route, optionally in combination with one or more other active agents, to the patient.

### BACKGROUND OF THE INVENTION

Type 2 diabetes mellitus is a common chronic and progressive disease arising from a complex pathophysiology involving the dual endocrine effects of insulin resistance and impaired insulin secretion with the consequence not meeting the required demands to maintain plasma glucose levels in the normal range. This leads to chronic hyperglycaemia and its associated micro- and macrovascular complications or chronic damages, such as e.g. diabetic nephropathy, retinopathy or neuropathy, or macrovascular (e.g. cardio- or cerebro-vascular) complications. The vascular disease component plays a significant role, but is not the only factor in the spectrum of diabetes associated disorders. The high frequency of complications leads to a significant reduction of life expectancy. Diabetes is currently the most frequent cause of adult-onset loss of vision, renal failure, and amputation in the Industrialised World because of diabetes induced complications and is associated with a two to five fold increase in cardiovascular disease risk.

Furthermore, diabetes (particularly type 2 diabetes) is often coexistent and interrelated with obesity and these two conditions together impose a particularly complex therapeutic challenge. Because of the effects of obesity on insulin resistance, weight loss and its maintenance is an important therapeutic objective in overweight or obese individuals with prediabetes, metabolic syndrome or diabetes. Studies have been demonstrated that weight reduction in subjects with type 2 diabetes is associated with decreased insulin resistance, improved measures of glycemia and lipemia, and reduced blood pressure. Maintenance of weight reduction over longer term is considered to improve glycemic control and prevent diabetic complications (e.g. reduction of risk for cardiovascular diseases or events). Thus, weight loss is recommended for all overweight or obese individuals who have or are at risk for diabetes. However, obese patients with type 2 diabetes have much greater difficulty losing weight and maintain the reduced weight than the general non-diabetic population.

Overweight may be defined as the condition wherein the individual has a body mass index (BMI) greater than or 25 kg/m² and less than 30 kg/m². The terms “overweight” and “pre-obese” are used interchangeably.

Obesity may be defined as the condition wherein the individual has a BMI equal to or greater than 30 kg/m². According to a WHO definition the term obesity may be categorized as follows: class I obesity is the condition wherein the BMI is equal to or greater than 30 kg/m² but lower than 35 kg/m²; class II obesity is the condition wherein the BMI is equal to or greater than 35 kg/m² but lower than 40 kg/m²; class III obesity is the condition wherein the BMI is equal to or greater than 40 kg/m². Obesity may include e.g. visceral or abdominal obesity.

Visceral obesity may be defined as the condition wherein a waist-to-hip ratio of greater than or equal to 1.0 in men and 0.8 in women is measured. It defines the risk for insulin resistance and the development of pre-diabetes.

Abdominal obesity may usually be defined as the condition wherein the waist circumference is >40 inches or 102 cm in men, and is >35 inches or 94 cm in women. With regard to a Japanese ethnicity or Japanese patients abdominal obesity may be defined as waist circumference ≧85 cm in men and ≧90 cm in women (see e.g. investigating committee for the diagnosis of metabolic syndrome in Japan).

The treatment of type 2 diabetes typically begins with diet and exercise, followed by oral antidiabetic monotherapy, and although conventional monotherapy may initially control blood glucose in some patients, it is however associated with a high secondary failure rate. The limitations of single-agent therapy for maintaining glycemic control may be overcome, at least in some patients, and for a limited period of time by combining multiple drugs to achieve reductions in blood glucose that cannot be sustained during long-term therapy with single agents. Available data support the conclusion that in most patients with type 2 diabetes current monotherapy will fail and treatment with multiple drugs will be required. But, because type 2 diabetes is a progressive disease, even patients with good initial responses to conventional combination therapy will eventually require an increase of the dosage or further treatment with insulin because the blood glucose level is very difficult to maintain stable for a long period of time. Although existing combination therapy has the potential to enhance glycemic control, it is not without limitations (especially with regard to long term efficacy). Further, traditional therapies may show an increased risk for side effects, such as hypoglycemia or weight gain, which may compromise their efficacy and acceptability.

Thus, for many patients, these existing drug therapies result in progressive deterioration in metabolic control despite treatment and do not sufficiently control metabolic status especially over long-term and thus fail to achieve and to maintain glycemic control in advanced or late stage type 2 diabetes, including diabetes with inadequate glycemic control despite conventional oral or non-oral antidiabetic medication.

Therefore, although intensive treatment of hyperglycemia can reduce the incidence of chronic damages, many patients with diabetes remain inadequately treated, partly because of limitations in long term efficacy, tolerability and dosing inconvenience of conventional antihyperglycemic therapies.

In addition, obesity, overweight or weight gain (e.g. as side or adverse effect of some conventional antidiabetic medications) further complicates the treatment of diabetes and its microvascular or macrovascular complications.

This high incidence of therapeutic failure is a major contributor to the high rate of long-term hyperglycemia-associated complications or chronic damages (including micro- and makrovascular complications such as e.g. diabetic nephropathy, retinopathy or neuropathy, or cerebro- or cardiovascular complications such as e.g. myocardial infarction, stroke or death) in patients with diabetes.

Oral antidiabetic drugs conventionally used in therapy (such as e.g. first- or second-line, and/or mono- or (initial or add-on) combination therapy) include, without being restricted thereto, metformin, sulphonylureas, thiazolidinediones, glinides and α-glucosidase inhibitors.

Non-oral (typically injected) antidiabetic drugs conventionally used in therapy (such as e.g. first- or second-line, and/or mono- or (initial or add-on) combination therapy) include, without being restricted thereto, GLP-1 or GLP-1 analogues, and insulin or insulin analogues.

However, the use of these conventional antidiabetic or antihyperglycemic agents can be associated with various adverse effects. For example, metformin can be associated with lactic acidosis or gastrointestinal side effects; sulfonylureas, glinides and insulin or insulin analogues can be associated with hypoglycemia and weight gain; thiazolidinediones can be associated with edema, bone fracture, weight gain and heart failure/cardiac effects; and alpha-glucosidase blockers and GLP-1 or GLP-1 analogues can be associated with gastrointestinal adverse effects (e.g. dyspepsia, flatulence or diarrhea, or nausea or vomiting).

Therefore, it remains a need in the art to provide efficacious, safe and tolerable antidiabetic therapies.

### SUMMARY OF THE INVENTION

The present invention relates to a method for treating and/or preventing metabolic diseases, especially type 2 diabetes mellitus, obesity, overweight, type 1 diabetes, LADA and/or conditions related thereto (e.g. diabetic complications), said method comprising or consisting essentially of administering a therapeutically effective amount of a certain DPP-4 inhibitor (particularly linagliptin) by subcutaneous or transdermal route, optionally in combination with one or more other active agents, to the patient.

The present invention further relates to pharmaceutical compositions or combinations comprising or consisting essentially of such active compounds, and to certain therapeutic uses thereof.

Further, the present invention relates to a method for improving glycemic control and/or preventing, reducing the risk of, slowing the progression of, delaying the onset or treating of complications of diabetes mellitus, such as micro- and macrovascular diseases (e.g. diabetic nephropathy, retinopathy or neuropathy, or cerebro- or cardiovascular complications such as e.g. myocardial infarction, stroke or vascular death or hospitalization), in a patient in need thereof (type 1 diabetes, LADA or, particularly, type 2 diabetes patient), said method comprising or consisting essentially of administering a therapeutically effective amount of a certain DPP-4 inhibitor (particularly linagliptin) by subcutaneous or transdermal route, optionally in combination with one or more other active agents, to the patient.

Further, the present invention relates to the use of a certain DPP-4 inhibitor (particularly linagliptin) for preparing a subcutaneous or transdermal pharmaceutical composition for treating and/or preventing metabolic diseases, for example type 2 diabetes mellitus, obesity, overweight, type 1 diabetes, LADA and/or conditions related thereto (e.g. diabetic complications).

Further, the present invention relates to the use of a certain DPP-4 inhibitor (particularly linagliptin) for preparing a pharmaceutical composition for subcutaneous or transdermal use in treating and/or preventing metabolic diseases, for example type 2 diabetes mellitus, obesity, overweight, type 1 diabetes, LADA and/or conditions related thereto (e.g. diabetic complications).

Further, the present invention relates to a certain DPP-4 inhibitor (particularly linagliptin) for subcutaneous or transdermal use in treating and/or preventing metabolic diseases, for example type 2 diabetes mellitus, obesity, overweight, type 1 diabetes, LADA and/or conditions related thereto (e.g. diabetic complications).

Furthermore, the present invention relates to a certain DPP-4 inhibitor (particularly linagliptin) for use in a method of treating and/or preventing a metabolic disease, especially type 2 diabetes mellitus, obesity, overweight, type 1 diabetes, LADA and/or conditions related thereto (e.g. diabetic complications), said method comprising or consisting essentially of administering subcutaneously (particularly by subcutaneous injection) a therapeutically effective amount (e.g. once daily, each other day, thrice weekly, twice weekly or once weekly) of the DPP-4 inhibitor (optionally in combination with one or more other active agents) to the patient in need thereof.

Furthermore, the present invention relates to a certain DPP-4 inhibitor (particularly linagliptin) for use in a method of treating and/or preventing a metabolic disease, especially type 2 diabetes mellitus, obesity, overweight, type 1 diabetes, LADA and/or conditions related thereto (e.g. diabetic complications), said method comprising or consisting essentially of administering transdermally a therapeutically effective amount (e.g. once daily, each other day, thrice weekly, twice weekly or once weekly) of the DPP-4 inhibitor (optionally in combination with one or more other active agents) to the patient in need thereof. Further, the present invention relates to a parenteral (preferably subcutaneous) delivery device, preferably a subcutaneous injection device, which may be with or without needle (e.g. a needle-based pen injector or a jet/needle-free injector), containing a certain DPP-4 inhibitor and, optionally, one or more pharmaceutically acceptable carriers and/or diluents.

Further, the present invention relates to a transdermal delivery device (e.g., a transdermal patch or gel) containing a certain DPP-4 inhibitor and, optionally, one or more pharmaceutically acceptable carriers and/or diluents.

The therapeutic and/or preventive methods or uses according to the present invention may involve the use of the DPP-4 inhibitor as mono- or combination therapy.

In one embodiment of this invention, the therapeutic and/or preventive methods or uses according to the present invention refer to the use of the DPP-4 inhibitor in monotherapy.

In another embodiment of this invention, the therapeutic and/or preventive methods or uses according to the present invention refer to the use of the DPP-4 inhibitor in combination therapy (e.g. dual or triple combination therapy).

In a further embodiment of this invention, the therapeutic and/or preventive methods or uses according to the present invention refer to the use of the DPP-4 inhibitor in mono- or combination therapy, with the proviso that combination therapy of the DPP-4 inhibitor with a long-acting insulin (basal insulin) is excluded.

Moreover, the present invention relates to a method for treating and/or preventing obesity or overweight or for reducing body weight in a subject (particularly human patient), said method comprising or consisting essentially of administering by subcutaneous or transdermal route an effective amount of a certain DPP-4 inhibitor (particularly linagliptin) and a GLP-1 analogue having a short half life (or to be administered at least twice daily) such as exendin (exendin-4 or exenatide) or, particularly, native GLP-1, to the subject in need thereof.

The present invention further relates to a subcutaneous or transdermal combination or composition containing a certain DPP-4 inhibitor (particularly linagliptin) and a GLP-1 (GLP-1 analogue or mimetic, or native GLP-1) having a short half life, particularly for reducing body weight or for treating obesity or overweight.

Further, the present invention relates to a pharmaceutical combination, composition or kit comprising or consisting essentially of a certain DPP-4 inhibitor (particularly linagliptin) and a GLP-1 analogue having a short half life (or to be administered at least twice daily) such as exendin (exendin-4 or exenatide) or, particularly, native GLP-1, e.g. for simultaneous and subcutaneous use of the active components, such as in treating and/or preventing obesity or overweight or for reducing body weight in a subject (particularly human patient).

Further, the present invention relates to the subcutaneous use of a certain DPP-4 inhibitor (particularly linagliptin) in combination with a GLP-1 analogue having a short half life (or to be administered at least twice daily) such as exendin (exendin-4 or exenatide) or, particularly, native GLP-1, for treating and/or preventing obesity or overweight or for reducing body weight.

Further, the present invention relates to use of a certain DPP-4 inhibitor (particularly linagliptin) and a GLP-1 analogue having a short half life (or to be administered at least twice daily) such as exendin (exendin-4 or exenatide) or, particularly, native GLP-1, for preparing a pharmaceutical composition for subcutaneous use in treating and/or preventing obesity or overweight or for reducing body weight.

Further, the present invention relates to a certain DPP-4 inhibitor (particularly linagliptin) and a GLP-1 analogue having a short half life (or to be administered at least twice daily) such as exendin (exendin-4 or exenatide) or, particularly, native GLP-1, each for subcutaneous use in treating and/or preventing obesity or overweight or for reducing body weight in a patient in need thereof (such as e.g. a type 2 diabetes mellitus, obesity, overweight, type 1 diabetes or LADA patient).

Further, the present invention relates to a combination of a certain DPP-4 inhibitor (particularly linagliptin) and a GLP-1 analogue having a short half life (or to be administered at least twice daily) such as exendin (exendin-4 or exenatide) or, particularly, native GLP-1, for simultaneous and subcutaneous use in treating and/or preventing obesity or overweight or for reducing body weight in a patient in need thereof (such as e.g. a type 2 diabetes mellitus, obesity, overweight, type 1 diabetes or LADA patient).

Moreover, the present invention relates to a method for treating and/or preventing metabolic diseases, especially type 2 diabetes mellitus, obesity, overweight, type 1 diabetes, LADA and/or conditions related thereto (e.g. diabetic complications) or for treating and/or preventing diabetes, obesity or overweight or for reducing body weight in a subject (particularly human patient), said method comprising or consisting essentially of administering by subcutaneous or transdermal route an effective amount of a certain DPP-4 inhibitor (particularly linagliptin) and an other (injectable) active agent which is a GLP-1 analogue having a short half life (or to be administered at least twice daily) such as exendin (exendin-4 or exenatide) or, particularly, native GLP-1, or amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), to the subject in need thereof.

Moreover, the present invention relates to a method for treating and/or preventing diabetes, obesity or overweight or for reducing body weight in a subject (particularly human patient), said method comprising or consisting essentially of administering by subcutaneous or transdermal route an effective amount of a certain DPP-4 inhibitor (particularly linagliptin) and an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), to the subject in need thereof.

The present invention further relates to a subcutaneous or transdermal combination or composition containing a certain DPP-4 inhibitor (particularly linagliptin) and an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), particularly for reducing body weight or for treating diabetes, obesity or overweight.

Further, the present invention relates to a pharmaceutical combination, composition or kit comprising or consisting essentially of a certain DPP-4 inhibitor (particularly linagliptin) and an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), e.g. for simultaneous and subcutaneous use of the active components, such as in treating and/or preventing diabetes, obesity or overweight or for reducing body weight in a subject (particularly human patient).

Further, the present invention relates to the subcutaneous use of a certain DPP-4 inhibitor (particularly linagliptin) in combination with an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), for treating and/or preventing diabetes, obesity or overweight or for reducing body weight.

Further, the present invention relates to use of a certain DPP-4 inhibitor (particularly linagliptin) and an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), for preparing a pharmaceutical composition for subcutaneous use in treating and/or preventing diabetes, obesity or overweight or for reducing body weight.

Further, the present invention relates to a certain DPP-4 inhibitor (particularly linagliptin) and an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination), each for subcutaneous use in treating and/or preventing diabetes, obesity or overweight or for reducing body weight in a patient in need thereof (such as e.g. a type 2 diabetes mellitus, obesity, overweight, type 1 diabetes or LADA patient).

Further, the present invention relates to a combination of a certain DPP-4 inhibitor (particularly linagliptin) and one or more other active agents selected from amylin or an amylin analogue, derivative or mimetic (such as e.g. pramlintide or davalintide), and leptin or a leptin analogue, derivative or mimetic (such as e.g. metreleptin), or a combination thereof (such as e.g. pramlintide/metreleptin combination), for simultaneous and subcutaneous use in treating and/or preventing diabetes, obesity or overweight or for reducing body weight in a patient in need thereof (such as e.g. a type 2 diabetes mellitus, obesity, overweight, type 1 diabetes or LADA patient).

### BRIEF DESCRIPTION OF THE DRAWINGS

FIG. 1 shows the DPP-4 activity in plasma after linagliptin subcutaneous (s.c.) dosing.

### DETAILED DESCRIPTION OF THE INVENTION

In an embodiment, the subject described herein is overweight or obese, e.g. with or without risk factors for or comorbidities such as diabetes mellitus, dyslipidemia, hypertension and/or metabolic syndrome.

In particular, the subject described herein is overweight or obese, e.g. with or without diabetes.

In another embodiment, the subject described herein is a subject having diabetes (e.g. type 1 or type 2 diabetes or LADA, particularly type 2 diabetes), e.g. with or without obesity or overweight.

In particular, the subject within this invention may be a human, e.g. a human child, a human adolescent or a human adult.

Diabetes patients within the meaning of this invention may include patients having obesity or overweight.

Obesity patients within the meaning of this invention may include, in one embodiment, patients with diabetes (particularly having type 2 diabetes, type 1 diabetes or LADA).

Obesity patients within the meaning of this invention may include, in another embodiment, patients without diabetes (particularly without type 1 or type 2 diabetes or LADA).

Further, within the therapy of type 2 diabetes, it is a need for treating the condition effectively, avoiding the complications inherent to the condition, and delaying disease progression.

Further, within the therapy of type 2 diabetes, it is a need for sustained improvements in diabetic phenotype, glycemic and/or metabolic control, and/or (blood) glucose profile (preferably over long-term and/or during chronic treatment).

Furthermore, it remains a need that antidiabetic treatments not only prevent the long-term complications often found in advanced stages of diabetes disease, but also are a therapeutic option in those diabetes patients who have developed or are at risk of developing complications, such as renal impairment.

Moreover, it remains a need to provide prevention or reduction of risk for adverse effects associated with conventional antidiabetic therapies.

Further, it remains a need in the art to provide efficacious, safe and tolerable therapies for obesity patients with or without diabetes, particularly for reducing body weight in such patients.

Further, within the management of the dual epidemic of diabetes and obesity (“diabesity”), it is an objective to find therapies which are safe, tolerable and effective in the treatment or prevention of these conditions together, particularly in achieving long term weight reduction and improving glycemic control.

The enzyme DPP-4 (dipeptidyl peptidase IV) also known as CD26 is a serine protease known to lead to the cleavage of a dipeptide from the N-terminal end of a number of proteins having at their N-terminal end a prolin or alanin residue. Due to this property DPP-4 inhibitors interfere with the plasma level of bioactive peptides including the peptide GLP-1 and are considered to be promising drugs for the treatment of diabetes mellitus.

For example, DPP-4 inhibitors and their uses are disclosed in WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769, WO2007/014886; WO 2004/050658, WO 2004/111051, WO 2005/058901, WO 2005/097798; WO 2006/068163, WO 2007/071738, WO 2008/017670; WO 2007/128721, WO 2007/128724, WO 2007/128761, or WO 2009/121945.

Inhibition of dipeptidyl peptidase 4 (DPP-4) is a novel treatment for type-2 diabetes. DPP-4 inhibition prevents the inactivation of glucagon-like peptide 1 (GLP-1) and therefore increases levels of active GLP-1. The activation of GLP-1 receptor by GLP-1 increases insulin secretion and reduces glucagon secretion, thereby improving glycemia. GLP-1 signals via a specific G protein-coupled receptor that activates the adenylyl cyclase pathway and it was shown that the carboxyl-terminal cytoplasmic tail of the GLP-1 receptor was phosphorylated at specific serine residues and phosphorylation correlates with GLP-1 receptor desensitivation. In line with that observation are clinical observations that GLP-1 analogues having a short half life such as exendin and consequently have to be administered twice daily are more efficient on body weight reduction than long-acting analogues such as liraglutide. In addition, recently was shown that the response to an oral glucose tolerance test (OGTT) was declined when another was performed shortly before.

Based on this data, GLP-1 analogues or mimetics (or GLP-1 receptor agonists in general) having a short half life (or to be administered subcuteanoulsy at least twice daily) such as exendin (exendin-4 or exenatide) or, particularly, native GLP-1, which has a half life of only 5 min in vivo, should have the most excessive effects on body weight reduction when the half life is prolonged by a DPP-4 inhibitor.

Linagliptin as a DPP-4 inhibitor only moderately increases GLP-1 and in contrast to GLP-1 analogues does not cause weight loss. Further, linagliptin is a DPP-4 inhibitor which can be administered subcutaneously. Therefore, the combination of linagliptin s.c. and a GLP-1 (GLP-1 analogue or mimetic, or a GLP-1 receptor agonist in general) having a short half life (or to be administered subcutaneously at least twice daily) such as exendin (exendin-4 or exenatide) or native GLP-1 is particularly suited and should have profound effects on body weight loss.

Within the context of this invention, short acting GLP-1, GLP-1 analogues, GLP-1 mimetics, GLP-1 receptor agonists, or the like are considered as interchangeable and refer to those of such agents having a short half life (or to be administered subcutaneously at least twice daily), such as e.g. exendin-4 or exenatide, or native GLP-1. All of these agents, as far as they exhibit the desired property and function, are contemplated and included within the scope of this invention.

Accordingly, a short acting GLP-1, GLP-1 analogue, GLP-1 mimetic, GLP-1 receptor agonist, or the like may be herein referred to as such agent having duration of action of <24 h, or having a short half life of about below 13 h, below 10 h, below 5 h, or below 2.5 h (e.g. about 2.4 h or even below), or to be administered subcutaneously at least twice daily, such as e.g. exenatide or native GLP-1.

In the monitoring of the treatment of diabetes mellitus the HbA1c value, the product of a non-enzymatic glycation of the haemoglobin B chain, is of exceptional importance. As its formation depends essentially on the blood sugar level and the life time of the erythrocytes the HbA1c in the sense of a “blood sugar memory” reflects the average blood sugar level of the preceding 4-12 weeks. Diabetic patients whose HbA1c level has been well controlled over a long time by more intensive diabetes treatment (i.e. <6.5% of the total haemoglobin in the sample) are significantly better protected from diabetic microangiopathy. The available treatments for diabetes can give the diabetic an average improvement in their HbA1c level of the order of 1.0-1.5%. This reduction in the HbA1C level is not sufficient in all diabetics to bring them into the desired target range of <7.0%, preferably <6.5% and more preferably <6% HbA1c.

Within the meaning of this invention, inadequate or insufficient glycemic control means in particular a condition wherein patients show HbA1c values above 6.5%, in particular above 7.0%, even more preferably above 7.5%, especially above 8%. An embodiment of patients with inadequate or insufficient glycemic control include, without being limited to, patients having a HbA1c value from 7.5 to 10% (or, in another embodiment, from 7.5 to 11%). A special sub-embodiment of inadequately controlled patients refers to patients with poor glycemic control including, without being limited, patients having a HbA1c value ≧9%.

Within glycemic control, in addition to improvement of the HbA1c level, other recommended therapeutic goals for type 2 diabetes mellitus patients are improvement of fasting plasma glucose (FPG) and of postprandial plasma glucose (PPG) levels to normal or as near normal as possible. Recommended desired target ranges of preprandial (fasting) plasma glucose are 70-130 mg/dL (or 90-130 mg/dL) or <110 mg/dL, and of two-hour postprandial plasma glucose are <180 mg/dL or <140 mg/dL.

In one embodiment, diabetes patients within the meaning of this invention may include patients who have not previously been treated with an antidiabetic drug (drug-naïve patients). Thus, in an embodiment, the therapies described herein may be used in naïve patients. In another embodiment, diabetes patients within the meaning of this invention may include patients with advanced or late stage type 2 diabetes mellitus (including patients with failure to conventional antidiabetic therapy), such as e.g. patients with inadequate glycemic control on one, two or more conventional oral and/or non-oral antidiabetic drugs as defined herein, such as e.g. patients with insufficient glycemic control despite (mono-)therapy with metformin, a thiazolidinedione (particularly pioglitazone), a sulphonylurea, a glinide, GLP-1 or GLP-1 analogue, insulin or insulin analogue, or an α-glucosidase inhibitor, or despite dual combination therapy with metformin/sulphonylurea, metformin/thiazolidinedione (particularly pioglitazone), sulphonylurea/α-glucosidase inhibitor, pioglitazone/sulphonylurea, metformin/insulin, pioglitazone/insulin or sulphonylurea/insulin. Thus, in an embodiment, the therapies described herein may be used in patients experienced with therapy, e.g. with conventional oral and/or non-oral antidiabetic mono- or dual or triple combination medication as mentioned herein.

A further embodiment of diabetic patients within the meaning of this invention refers to patients ineligible for metformin therapy including
patients for whom metformin therapy is contraindicated, e.g. patients having one or more contraindications against metformin therapy according to label, such as for example patients with at least one contraindication selected from:
   renal disease, renal impairment or renal dysfunction (e.g., as specified by product information of locally approved metformin),
   dehydration,
   unstable or acute congestive heart failure,
   acute or chronic metabolic acidosis, and
   hereditary galactose intolerance;
      and
patients who suffer from one or more intolerable side effects attributed to metformin, particularly gastrointestinal side effects associated with metformin, such as for example patients suffering from at least one gastrointestinal side effect selected from:
   nausea,
   vomiting,
   diarrhoea,
   intestinal gas, and
   severe abdominal discomfort.

A further embodiment of the diabetes patients which may be amenable to the therapies of this invention may include, without being limited, those diabetes patients for whom normal metformin therapy is not appropriate, such as e.g. those diabetes patients who need reduced dose metformin therapy due to reduced tolerability, intolerability or contraindication against metformin or due to (mildly) impaired/reduced renal function (including elderly patients, such as e.g. ≧60-65 years).

A further embodiment of diabetic patients within the meaning of this invention refers to patients having renal disease, renal dysfunction, or insufficiency or impairment of renal function (including mild, moderate and severe renal impairment), e.g. as suggested by elevated serum creatinine levels (e.g. serum creatinine levels above the upper limit of normal for their age, e.g. ≧130-150 μmol/l, or ≧1.5 mg/dl (≧136 μmol/l) in men and ≧1.4 mg/dl (≧124 μmol/l) in women) or abnormal creatinine clearance (e.g. glomerular filtration rate (GFR) ≦30-60 ml/min).

In this context, for more detailed example, mild renal impairment may be e.g. suggested by a creatinine clearance of 50-80 ml/min (approximately corresponding to serum creatine levels of ≦1.7 mg/dL in men and ≦1.5 mg/dL in women); moderate renal impairment may be e.g. suggested by a creatinine clearance of 30-50 ml/min (approximately corresponding to serum creatinine levels of >1.7 to ≦3.0 mg/dL in men and >1.5 to ≦2.5 mg/dL in women); and severe renal impairment may be e.g. suggested by a creatinine clearance of <30 ml/min (approximately corresponding to serum creatinine levels of >3.0 mg/dL in men and >2.5 mg/dL in women). Patients with end-stage renal disease require dialysis (e.g. hemodialysis or peritoneal dialysis).

For other more detailed example, patients with renal disease, renal dysfunction or renal impairment include patients with chronic renal insufficiency or impairment, which can be stratified according to glomerular filtration rate (GFR, ml/min/1.73 m²) into 5 disease stages: stage 1 characterized by normal GFR ≧90 plus either persistent albuminuria or known structural or hereditary renal disease; stage 2 characterized by mild reduction of GFR (GFR 60-89) describing mild renal impairment; stage 3 characterized by moderate reduction of GFR (GFR 30-59) describing moderate renal impairment; stage 4 characterized by severe reduction of GFR (GFR 15-29) describing severe renal impairment; and terminal stage 5 characterized by requiring dialysis or GFR <15 describing established kidney failure (end-stage renal disease, ESRD).

A further embodiment of diabetic patients within the meaning of this invention refers to type 2 diabetes patients with or at risk of developing renal complications, such as diabetic nephropathy (including chronic and progressive renal insufficiency, albuminuria, proteinuria, fluid retention in the body (edema) and/or hypertension).

In a further embodiment, patients within the present invention may include type 1 diabetes, LADA or, particularly, type 2 diabetes patients, with or without obesity or overweight.

Within the scope of the present invention it has now been found that certain DPP-4 inhibitors as defined herein as well as pharmaceutical combinations, compositions, uses or methods according to this invention of these DPP-4 inhibitors and, optionally, one or more other active agents (such as e.g short-acting GLP-1 analogues/mimetics or GLP-1 receptor agonists, e.g. GLP-1 analogues having short half life such as e.g. exendin-4 or exenatide or native GLP-1) as defined herein have properties, which make them suitable for the purpose of this invention and/or for fulfilling one or more of above needs.

Examples of such metabolic disorders or diseases amenable by the therapy of this invention may include, without being limited to, type 1 diabetes, type 2 diabetes, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, postprandial lipemia, hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome.

The present invention further relates to at least one of the following methods:
preventing, slowing the progression of, delaying or treating a metabolic disorder or disease, such as e.g. type 1 diabetes mellitus, type 2 diabetes mellitus, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, postprandial lipemia, hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome;
improving and/or maintaining glycemic control and/or for reducing of fasting plasma glucose, of postprandial plasma glucose, of postabsorptive plasma glucose and/or of glycosylated hemoglobin HbA1c;
preventing, slowing, delaying or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying or treating of complications of diabetes mellitus such as micro- and macrovascular diseases, such as nephropathy, micro- or macroalbuminuria, proteinuria, retinopathy, cataracts, neuropathy, learning or memory impairment, neurodegenerative or cognitive disorders, cardio- or cerebrovascular diseases, tissue ischaemia, diabetic foot or ulcus, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke;
reducing body weight and/or body fat and/or liver fat and/or intra-myocellular fat or preventing an increase in body weight and/or body fat and/or liver fat and/or intra-myocellular fat or facilitating a reduction in body weight and/or body fat and/or liver fat and/or intra-myocellular fat;
preventing, slowing, delaying or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing, delaying or treating non alcoholic fatty liver disease (NAFLD) including hepatic steatosis, non-alcoholic steatohepatitis (NASH) and/or liver fibrosis (such as e.g. preventing, slowing the progression, delaying, attenuating, treating or reversing hepatic steatosis, (hepatic) inflammation and/or an abnormal accumulation of liver fat);
preventing, slowing the progression of, delaying or treating type 2 diabetes with failure to conventional antidiabetic mono- or combination therapy;
achieving a reduction in the dose of conventional antidiabetic medication required for adequate therapeutic effect;
reducing the risk for adverse effects associated with conventional antidiabetic medication (e.g. hypoglycemia or weight gain); and/or
maintaining and/or improving the insulin sensitivity and/or for treating or preventing hyperinsulinemia and/or insulin resistance;
in a patient in need thereof (such as e.g. a patient as described herein), said method comprising administering subcutaneously or transdermally a therapeutically effective amount of a DPP-4 inhibitor as defined herein (particularly linagliptin, such as e.g. in a subcutaneous amount of 0.3-10 mg or 0.1-30 mg, preferably from 1 to 5 mg or from 1 to 10 mg, e.g. 2.5 mg or 5 mg per day) and a GLP-1 analogue having a short half life (or to be administered at least twice daily) such as exendin (exendin-4 or exenatide) or, particularly, native GLP-1, to the patient.

The present invention further relates to a method for treating and/or preventing obesity or overweight or for reducing body weight in a subject (particularly human patient in need thereof), said method comprising administering subcutaneously or transdermally an effective amount of a DPP-4 inhibitor as defined herein (particularly linagliptin, such as e.g. in a subcutaneous amount of 0.3-10 mg or 0.1-30 mg, preferably from 1 to 5 mg or from 1 to 10 mg, e.g. 2.5 mg or 5 mg per day) and a GLP-1 analogue having a short half life (or to be administered at least twice daily) such as exendin (exendin-4 or exenatide) or, particularly, native GLP-1, to the subject.

The present invention further relates to at least one of the following methods:
preventing, slowing the progression of, delaying or treating a metabolic disorder or disease, such as e.g. type 1 diabetes mellitus, type 2 diabetes mellitus, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, postprandial lipemia, hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome;
improving and/or maintaining glycemic control and/or for reducing of fasting plasma glucose, of postprandial plasma glucose, of postabsorptive plasma glucose and/or of glycosylated hemoglobin HbA1c;
preventing, slowing, delaying or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying or treating of complications of diabetes mellitus such as micro- and macrovascular diseases, such as nephropathy, micro- or macroalbuminuria, proteinuria, retinopathy, cataracts, neuropathy, learning or memory impairment, neurodegenerative or cognitive disorders, cardio- or cerebrovascular diseases, tissue ischaemia, diabetic foot or ulcus, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke;
reducing body weight and/or body fat and/or liver fat and/or intra-myocellular fat or preventing an increase in body weight and/or body fat and/or liver fat and/or intra-myocellular fat or facilitating a reduction in body weight and/or body fat and/or liver fat and/or intra-myocellular fat;
preventing, slowing, delaying or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing, delaying or treating non alcoholic fatty liver disease (NAFLD) including hepatic steatosis, non-alcoholic steatohepatitis (NASH) and/or liver fibrosis (such as e.g. preventing, slowing the progression, delaying, attenuating, treating or reversing hepatic steatosis, (hepatic) inflammation and/or an abnormal accumulation of liver fat);
preventing, slowing the progression of, delaying or treating type 2 diabetes with failure to conventional antidiabetic mono- or combination therapy;
achieving a reduction in the dose of conventional antidiabetic medication required for adequate therapeutic effect;
reducing the risk for adverse effects associated with conventional antidiabetic medication (e.g. hypoglycemia or weight gain); and/or
maintaining and/or improving the insulin sensitivity and/or for treating or preventing hyperinsulinemia and/or insulin resistance;
in a patient in need thereof (such as e.g. a patient as described herein), said method comprising administering subcutaneously or transdermally a therapeutically effective amount of a DPP-4 inhibitor as defined herein (particularly linagliptin, such as e.g. in a subcutaneous amount of 0.3-10 mg or 0.1-30 mg, preferably from 1 to 5 mg or from 1 to 10 mg, e.g. 2.5 mg or 5 mg per day), optionally in combination with one or more other therapeutic agents as described herein, to the patient.

Further, the present invention relates to a pharmaceutical composition according to this invention comprising

a DPP-4 inhibitor (preferably linagliptin) as defined herein, and, optionally,
a GLP-1 (GLP-1 mimetic, exenatide or native GLP-1) having a short half life as defined herein;
and, optionally, one or more pharmaceutically acceptable carriers and/or diluents, said composition being for subcutaneous administration to the patient in need thereof, e.g. by injection.

Further, the present invention relates to a combination, kit or pharmaceutical composition according to this invention comprising

a DPP-4 inhibitor (preferably linagliptin) as defined herein, and, optionally, an other active agent which is a GLP-1 (GLP-1 mimetic, exenatide or native GLP-1) having a short half life as defined herein, amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination);
and, optionally, one or more pharmaceutically acceptable carriers and/or diluents, said combination, kit or composition being for subcutaneous (separate, simultaneous or sequential) administration of the active components to the patient in need thereof, e.g. by injection of any or all components.

Other aspects of the present invention become apparent to the skilled person from the foregoing and following remarks (including the examples and claims).

In particular embodiments, the aspects of the present invention, in particular the pharmaceutical compounds, compositions, combinations, methods and uses, refer to DPP-4 inhibitors and/or GLP-1 (GLP-1 mimetic or native GLP-1) having a short half life as defined hereinbefore and hereinafter.

In other embodiments, the aspects of the present invention, in particular the pharmaceutical compounds, compositions, combinations, methods and uses, refer to DPP-4 inhibitors and/or an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination).

In other embodiments, the aspects of the present invention, in particular the pharmaceutical compounds, compositions, combinations, methods and uses, refer to DPP-4 inhibitors and/or an other active agent which is amylin or an amylin analogue, derivative or mimetic, particularly pramlintide or davalintide, or a pramlintide/metreleptin combination.

In other embodiments, the aspects of the present invention, in particular the pharmaceutical compounds, compositions, combinations, methods and uses, refer to DPP-4 inhibitors and/or an other active agent which is leptin or a leptin analogue, derivative or mimetic (such as e.g. metreleptin), or a pramlintide/metreleptin combination.

A DPP-4 inhibitor within the meaning of the present invention includes, without being limited to, any of those DPP-4 inhibitors mentioned hereinabove and herein below, preferably subcutaneously active DPP-4 inhibitors.

An embodiment of this invention refers to a DPP-4 inhibitor for use in the treatment and/or prevention of metabolic diseases (particularly type 2 diabetes mellitus) in type 2 diabetes patients, wherein said patients further suffering from renal disease, renal dysfunction or renal impairment, particularly characterized in that said DPP-4 inhibitor is administered to said patients in the same dose levels as to patients with normal renal function, thus e.g. said DPP-4 inhibitor does not require downward dosing adjustment for impaired renal function.

For example, a DPP-4 inhibitor according to this invention (especially one which may be suited for patients with impaired renal function) may be such an oral DPP-4 inhibitor, which and whose active metabolites have preferably a relatively wide (e.g. about >100 fold) therapeutic window and/or, especially, that are primarily eliminated via hepatic metabolism or biliary excretion (preferably without adding additional burden to the kidney).

In more detailed example, a DPP-4 inhibitor according to this invention (especially one which may be suited for patients with impaired renal function) may be such an orally administered DPP-4 inhibitor, which has a relatively wide (e.g. >100 fold) therapeutic window (preferably a safety profile comparable to placebo) and/or which fulfils one or more of the following pharmacokinetic properties (preferably at its therapeutic oral dose levels):
The DPP-4 inhibitor is substantially or mainly excreted via the liver (e.g. >80% or even >90% of the administered oral dose), and/or for which renal excretion represents no substantial or only a minor elimination pathway (e.g. <10%, preferably <7%, of the administered oral dose measured, for example, by following elimination of a radiolabelled carbon (¹⁴C) substance oral dose);
The DPP-4 inhibitor is excreted mainly unchanged as parent drug (e.g. with a mean of >70%, or >80%, or, preferably, 90% of excreted radioactivity in urine and faeces after oral dosing of radiolabelled carbon (¹⁴C) substance), and/or which is eliminated to a non-substantial or only to a minor extent via metabolism (e.g. <30%, or <20%, or, preferably, 10%);
The (main) metabolite(s) of the DPP-4 inhibitor is/are pharmacologically inactive. Such as e.g. the main metabolite does not bind to the target enzyme DPP-4 and, optionally, it is rapidly eliminated compared to the parent compound (e.g. with a terminal half-life of the metabolite of ≦20 h, or, preferably, ≦about 16 h, such as e.g. 15.9 h).

In one embodiment, the (main) metabolite in plasma (which may be pharmacologically inactive) of a DPP-4 inhibitor having a 3-amino-piperidin-1-yl substituent is such a derivative where the amino group of the 3-amino-piperidin-1-yl moiety is replaced by a hydroxyl group to form the 3-hydroxy-piperidin-1-yl moiety (e.g. the 3-(S)-hydroxy-piperidin-1-yl moiety, which is formed by inversion of the configuration of the chiral center).

Further properties of a DPP-4 inhibitor according to this invention may be one or more of the following: Rapid attainment of steady state (e.g. reaching steady state plasma levels (>90% of the steady state plasma concentration) between second and fifth day of treatment with therapeutic oral dose levels), little accumulation (e.g. with a mean accumulation ratio R_{A,AUC}≦1.4 with therapeutic oral dose levels), and/or preserving a long-lasting effect on DPP-4 inhibition, preferably when used once-daily (e.g. with almost complete (>90%) DPP-4 inhibition at therapeutic oral dose levels, >80% inhibition over a 24 h interval after once-daily intake of therapeutic oral drug dose), significant decrease in 2 h postprandial blood glucose excursions by ≧80% (already on first day of therapy) at therapeutic dose levels, and cumulative amount of unchanged parent compound excreted in urine on first day being below 1% of the administered dose and increasing to not more than about 3-6% in steady state.

Thus, for example, a DPP-4 inhibitor according to this invention may be characterized in that said DPP-4 inhibitor has a primarily non-renal route of excretion, i.e. said DPP-4 inhibitor is excreted to a non-substantial or only to a minor extent (e.g. <10%, preferably <7%, e.g. about 5%, of administered oral dose, preferably of oral therapeutic dose) via the kidney (measured, for example, by following elimination of a radiolabelled carbon (¹⁴C) substance oral dose).

Further, a DPP-4 inhibitor according to this invention may be characterized in that said DPP-4 inhibitor is excreted substantially or mainly via the liver or faeces (measured, for example, by following elimination of a radiolabelled carbon (¹⁴C) substance oral dose).

Further, a DPP-4 inhibitor according to this invention may be characterized in that said DPP-4 inhibitor is excreted mainly unchanged as parent drug (e.g. with a mean of >70%, or >80%, or, preferably, 90% of excreted radioactivity in urine and faeces after oral dosing of radiolabelled carbon (¹⁴C) substance),

said DPP-4 inhibitor is eliminated to a non-substantial or only to a minor extent via metabolism, and/or
the main metabolite of said DPP-4 inhibitor is pharmacologically inactive or has a relatively wide therapeutic window.

Further, a DPP-4 inhibitor according to this invention may be characterized in that said DPP-4 inhibitor does not significantly impair glomerular and/or tubular function of a type 2 diabetes patient with chronic renal insufficiency (e.g. mild, moderate or severe renal impairment or end stage renal disease), and/or

said DPP-4 inhibitor trough levels in the blood plasma of type 2 diabetes patients with mild or moderate renal impairment are comparable to the levels in patients with normal renal function, and/or
said DPP-4 inhibitor does not require to be dose-adjusted in a type 2 diabetes patient with impaired renal function (e.g. mild, moderate or severe renal impairment or end stage renal disease, preferably regardless of the stage of renal impairment).

Further, a DPP-4 inhibitor according to this invention may be characterized in that said DPP-4 inhibitor provides its minimally effective dose at that dose that results in >50% inhibition of DPP-4 activity at trough (24 h after last dose) in >80% of patients, and/or said DPP-4 inhibitor provides its fully therapeutic dose at that dose that results in >80% inhibition of DPP-4 activity at trough (24 h after last dose) in >80% of patients.

Further, a DPP-4 inhibitor according to this invention may be characterized in that being suitable for use in type 2 diabetes patients who are with diagnosed renal impairment and/or who are at risk of developing renal complications, e.g. patients with or at risk of diabetic nephropathy (including chronic and progressive renal insufficiency, albuminuria, proteinuria, fluid retention in the body (edema) and/or hypertension).

In a first embodiment (embodiment A), a DPP-4 inhibitor in the context of the present invention is any DPP-4 inhibitor of formula (I)

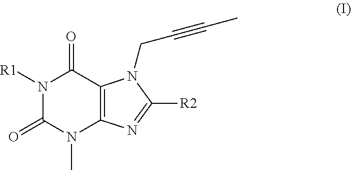

or formula (II)

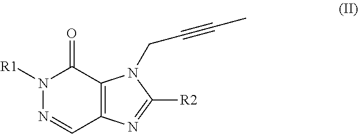

or formula (III)

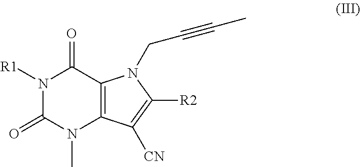

or formula (IV)

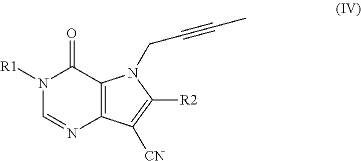

wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3-cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino,
or its pharmaceutically acceptable salt.

Regarding the first embodiment (embodiment A), preferred DPP-4 inhibitors are any or all of the following compounds and their pharmaceutically acceptable salts:

### 1-[(4-methyl-quinazolin-2-yl)methyl]-3-methyl-7-(2-butyn-1-yl)-8-(3-(R)-amino-piperidin-1-yl)-xanthine (compare WO 2004/018468, example 2(142))

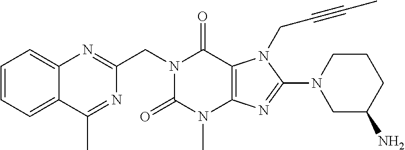 [SMILES: CC#CCn1c(N2CCC[C@@H](N)C2)nc2c1c(=O)n(Cc1nc(C)c3ccccc3n1)c(=O)n2C]

### 1-[([1,5]naphthyridin-2-yl)methyl]-3-methyl-7-(2-butyn-1-yl)-8-((R)-3-amino-piperidin-1-yl)-xanthine (compare WO 2004/018468, example 2(252))

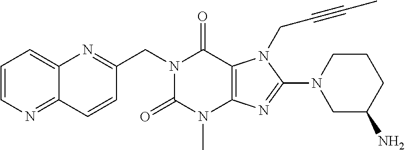 [SMILES: CC#CCn1c(N2CCC[C@@H](N)C2)nc2c1c(=O)n(Cc1ccc3ncccc3n1)c(=O)n2C]

### 1-[(Quinazolin-2-yl)methyl]-3-methyl-7-(2-butyn-1-yl)-8-((R)-3-amino-piperidin-1-yl)-xanthine (compare WO 2004/018468, example 2(80))

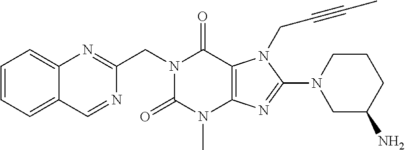 [SMILES: CC#CCn1c(N2CCC[C@@H](N)C2)nc2c1c(=O)n(Cc1ncc3ccccc3n1)c(=O)n2C]

### 2-((R)-3-Amino-piperidin-1-yl)-3-(but-2-yinyl)-5-(4-methyl-quinazolin-2-ylmethyl)-3,5-dihydro-imidazo[4,5-d]pyridazin-4-one (compare WO 2004/050658, example 136)

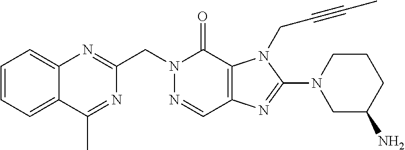 [SMILES: CC#CCn1c(N2CCC[C@@H](N)C2)nc2cnn(Cc3nc(C)c4ccccc4n3)c(=O)c21]

### 1-[(4-Methyl-quinazolin-2-yl)methyl]-3-methyl-7-(2-butyin-1-yl)-8-[(2-amino-2-methyl-propyl)-methylamino]-xanthine (compare WO 2006/029769, example 2(1))

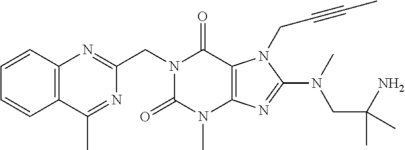 [SMILES: CC#CCn1c(N(C)CC(C)(C)N)nc2c1c(=O)n(Cc1nc(C)c3ccccc3n1)c(=O)n2C]

### 1-[(3-Cyano-quinolin-2-yl)methyl]-3-methyl-7-(2-butyn-1-yl)-8-((R)-3-amino-piperidin-1-yl)-xanthine (compare WO 2005/085246, example 1(30))

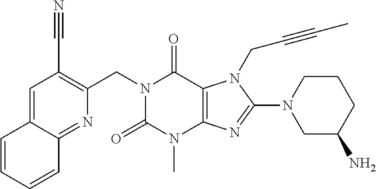 [SMILES: CC#CCn1c(N2CCC[C@@H](N)C2)nc2c1c(=O)n(Cc1nc3ccccc3cc1C#N)c(=O)n2C]

### 1-(2-Cyano-benzyl)-3-methyl-7-(2-butyn-1-yl)-8-((R)-3-amino-piperidin-1-yl)-xanthine (compare WO 2005/085246, example 1(39))

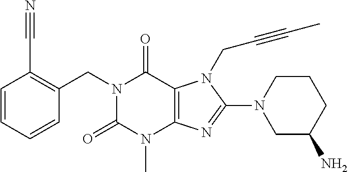 [SMILES: CC#CCn1c(N2CCC[C@@H](N)C2)nc2c1c(=O)n(Cc1ccccc1C#N)c(=O)n2C]

### 1-[(4-Methyl-quinazolin-2-yl)methyl]-3-methyl-7-(2-butyn-1-yl)-8-[(S)-(2-amino-propyl)-methylamino]-xanthine (compare WO 2006/029769, example 2(4))

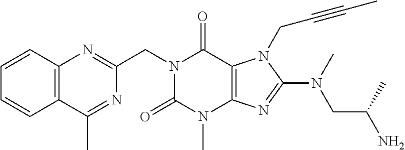 [SMILES: CC#CCn1c(N(C)C[C@H](C)N)nc2c1c(=O)n(Cc1nc(C)c3ccccc3n1)c(=O)n2C]

### 1-[(3-Cyano-pyridin-2-yl)methyl]-3-methyl-7-(2-butyn-1-yl)-8-((R)-3-amino-piperidin-1-yl)-xanthine (compare WO 2005/085246, example 1(52))

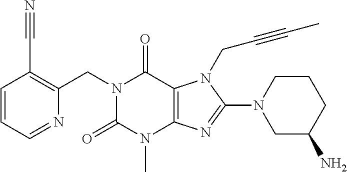 [SMILES: CC#CCn1c(N2CCC[C@@H](N)C2)nc2c1c(=O)n(Cc1ncccc1C#N)c(=O)n2C]

### 1-[(4-Methyl-pyrimidin-2-yl)methyl]-3-methyl-7-(2-butyn-1-yl)-8-((R)-3-amino-piperidin-1-yl)-xanthine (compare WO 2005/085246, example 1(81))

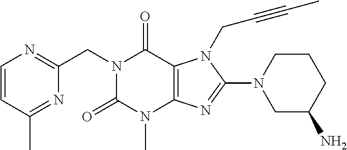 [SMILES: CC#CCn1c(N2CCC[C@@H](N)C2)nc2c1c(=O)n(Cc1nccc(C)n1)c(=O)n2C]

### 1-[(4,6-Dimethyl-pyrimidin-2-yl)methyl]-3-methyl-7-(2-butyn-1-yl)-8-((R)-3-amino-piperidin-1-yl)-xanthine (compare WO 2005/085246, example 1(82))

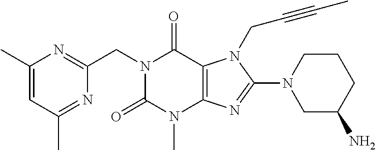 [SMILES: CC#CCn1c(N2CCC[C@@H](N)C2)nc2c1c(=O)n(Cc1nc(C)cc(C)n1)c(=O)n2C]

### 1-[(Quinoxalin-6-yl)methyl]-3-methyl-7-(2-butyn-1-yl)-8-((R)-3-amino-piperidin-1-yl)-xanthine (compare WO 2005/085246, example 1(83))

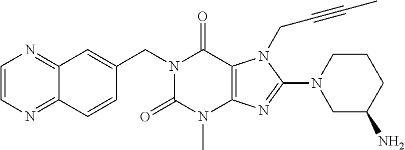 [SMILES: CC#CCn1c(N2CCC[C@@H](N)C2)nc2c1c(=O)n(Cc1ccc3nccnc3c1)c(=O)n2C]

These DPP-4 inhibitors are distinguished from structurally comparable DPP-4 inhibitors, as they combine exceptional potency and a long-lasting effect with favourable pharmacological properties, receptor selectivity and a favourable side-effect profile or bring about unexpected therapeutic advantages or improvements when combined with other pharmaceutical active substances. Their preparation is disclosed in the publications mentioned.

A more preferred DPP-4 inhibitor among the abovementioned DPP-4 inhibitors of embodiment A of this invention is 1-[(4-methyl-quinazolin-2-yl)methyl]-3-methyl-7-(2-butyn-1-yl)-8-(3-(R)-amino-piperidin-1-yl)-xanthine, particularly the free base thereof (which is also known as linagliptin or BI 1356).

A particularly preferred DPP-4 inhibitor within the present invention is linagliptin. The term “linagliptin” as employed herein refers to linagliptin or a pharmaceutically acceptable salt thereof, including hydrates and solvates thereof, and crystalline forms thereof, preferably linagliptin refers to 1-[(4-methyl-quinazolin-2-yl)methyl]-3-methyl-7-(2-butyn-1-yl)-8-(3-(R)-amino-piperidin-1-yl)-xanthine. Crystalline forms are described in WO 2007/128721. Methods for the manufacture of linagliptin are described in the patent applications WO 2004/018468 and WO 2006/048427 for example. Linagliptin is distinguished from structurally comparable DPP-4 inhibitors, as it combines exceptional potency and a long-lasting effect with favourable pharmacological properties, receptor selectivity and a favourable side-effect profile or bring about unexpected therapeutic advantages or improvements in mono- or dual or triple combination therapy.

For avoidance of any doubt, the disclosure of each of the foregoing and following documents cited above in connection with the specified DPP-4 inhibitors is specifically incorporated herein by reference in its entirety.

Within this invention it is to be understood that the combinations, compositions or combined uses according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The present invention also provides a kit-of-parts or combination therapeutic product comprising

a) a pharmaceutical composition comprising a DPP-4 inhibitor (preferably linagliptin) as defined herein, optionally together with one or more pharmaceutically acceptable carriers and/or diluents, and
b) a pharmaceutical composition comprising a GLP-1 (GLP-1 mimetic, exenatide or native GLP-1) having a short half life as defined herein, optionally together with one or more pharmaceutically acceptable carriers and/or diluents.

The present invention also provides a kit comprising

a) a DPP-4 inhibitor (preferably linagliptin) as defined herein, and
b) a GLP-1 (GLP-1 mimetic, exenatide or native GLP-1) having a short half life as defined herein,
and, optionally, instructions directing use of the DPP-4 inhibitor and the GLP-1 having a short half life in combination (e.g. simultaneously), e.g. for a purpose of this invention, such as e.g. for reducing body weight.

The present invention also provides a pharmaceutical composition or fixed dose combination comprising

a) a DPP-4 inhibitor (preferably linagliptin) as defined herein, and
b) a GLP-1 (GLP-1 mimetic, exenatide or native GLP-1) having a short half life as defined herein;
and, optionally, one or more pharmaceutically acceptable carriers and/or diluents.

The present invention also provides a transdermal or subcutaneous (injectable) pharmaceutical composition, delivery system or device for systemic use comprising

a) a DPP-4 inhibitor (preferably linagliptin) as defined herein, and, optionally,
b) a GLP-1 (GLP-1 mimetic, exenatide or native GLP-1) having a short half life as defined herein;
and, optionally, one or more pharmaceutically acceptable carriers and/or diluents.

In another embodiment, the present invention relates to a pharmaceutical composition or fixed dose combination consisting essentially of

a) a DPP-4 inhibitor (preferably linagliptin) as defined herein, and
b) a GLP-1 (GLP-1 mimetic, exenatide or native GLP-1) having a short half life as defined herein;
and, optionally, one or more pharmaceutically acceptable carriers and/or diluents.

In another embodiment, the present invention also provides a transdermal or subcutaneous (injectable) pharmaceutical composition, delivery system or device for systemic use consisting essentially of

a) a DPP-4 inhibitor (preferably linagliptin) as defined herein, and,
b) a GLP-1 (GLP-1 mimetic, exenatide or native GLP-1) having a short half life as defined herein;
and, optionally, one or more pharmaceutically acceptable carriers and/or diluents.

In another embodiment, the present invention relates to a pharmaceutical composition or fixed dose combination consisting essentially of

a) a DPP-4 inhibitor (preferably linagliptin) as defined herein, and
b) an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination);
and, optionally, one or more pharmaceutically acceptable carriers and/or diluents.

In another embodiment, the present invention also provides a transdermal or subcutaneous (injectable) pharmaceutical composition, delivery system or device for systemic use consisting essentially of

a) a DPP-4 inhibitor (preferably linagliptin) as defined herein, and,
b) an other active agent which is amylin or an amylin analogue, derivative or mimetic (such as e.g. pramlintide or davalintide), or leptin or a leptin analogue, derivative or mimetic (such as e.g. metreleptin), or a combination thereof (such as e.g. pramlintide/metreleptin combination);
and, optionally, one or more pharmaceutically acceptable carriers and/or diluents.

The combined administration of this invention may take place by administering the active components or ingredients together, such as e.g. by administering them simultaneously in one single or in two separate formulations or dosage forms. Alternatively, the administration may take place by administering the active components or ingredients sequentially, such as e.g. successively in two separate formulations or dosage forms.

For the combination therapy of this invention the active components or ingredients may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination. In one embodiment, for the combination therapy according to this invention the DPP-4 inhibitor and the GLP-1 having a short half life are administered in different formulations or different dosage forms. In another embodiment, for the combination therapy according to this invention the DPP-4 inhibitor and the GLP-1 having a short half life are administered in the same formulation or in the same dosage form. In a further embodiment, for the combination therapy according to this invention the DPP-4 inhibitor and the GLP-1 having a short half life are administered simultaneously. In a further embodiment, for the combination therapy according to this invention the DPP-4 inhibitor and the GLP-1 having a short half life are each administered subcutaneously. In a further embodiment, for the combination therapy according to this invention the DPP-4 inhibitor and the GLP-1 having a short half life are administered simultaneously and each subcutaneously.

Unless otherwise noted, combination therapy may refer to first line, second line or third line therapy, or initial or add-on combination therapy or replacement therapy.

With respect to embodiment A, the methods of synthesis for the DPP-4 inhibitors according to embodiment A of this invention are known to the skilled person. Advantageously, the DPP-4 inhibitors according to embodiment A of this invention can be prepared using synthetic methods as described in the literature. Thus, for example, purine derivatives of formula (I) can be obtained as described in WO 2002/068420, WO 2004/018468, WO 2005/085246, WO 2006/029769 or WO 2006/048427, the disclosures of which are incorporated herein. Purine derivatives of formula (II) can be obtained as described, for example, in WO 2004/050658 or WO 2005/110999, the disclosures of which are incorporated herein. Purine derivatives of formula (III) and (IV) can be obtained as described, for example, in WO 2006/068163, WO 2007/071738 or WO 2008/017670, the disclosures of which are incorporated herein. The preparation of those DPP-4 inhibitors, which are specifically mentioned hereinabove, is disclosed in the publications mentioned in connection therewith. Polymorphous crystal modifications and formulations of particular DPP-4 inhibitors are disclosed in WO 2007/128721 and WO 2007/128724, respectively, the disclosures of which are incorporated herein in their entireties. Formulations of particular DPP-4 inhibitors with metformin or other combination partners are described in WO 2009/121945, the disclosure of which is incorporated herein in its entirety.

Typical dosage strengths of the dual fixed combination (tablet) of linagliptin/metformin IR (immediate release) are 2.5/500 mg, 2.5/850 mg and 2.5/1000 mg, which may be administered 1-3 times a day, particularly twice a day.

Typical dosage strengths of the dual fixed combination (tablet) of linagliptin/metformin XR (extended release) are 5/500 mg, 5/1000 mg and 5/1500 mg (each one tablet) or 2.5/500 mg, 2.5/750 mg and 2.5/1000 mg (each two tablets), which may be administered 1-2 times a day, particularly once a day, preferably to be taken in the evening with meal.

The present invention further provides a DPP-4 inhibitor as defined herein for use in (add-on or initial) combination therapy with metformin (e.g. in a total daily amount from 500 to 2000 mg metformin hydrochloride, such as e.g. 500 mg, 850 mg or 1000 mg once or twice daily).

For pharmaceutical application in warm-blooded vertebrates, particularly humans, the compounds of this invention are usually used in dosages from 0.001 to 100 mg/kg body weight, preferably at 0.01-15 mg/kg or 0.1-15 mg/kg, in each case 1 to 4 times a day. For this purpose, the compounds, optionally combined with other active substances, may be incorporated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof into conventional galenic preparations such as plain or coated tablets, capsules, powders, suspensions or suppositories.

The pharmaceutical compositions according to this invention comprising the DPP-4 inhibitors as defined herein are thus prepared by the skilled person using pharmaceutically acceptable formulation excipients as described in the art and appropriate for the desired route of administration. Examples of such excipients include, without being restricted to diluents, binders, carriers, fillers, lubricants, flow promoters, crystallisation retardants, disintegrants, solubilizers, colorants, pH regulators, surfactants and emulsifiers.

Oral formulations or dosage forms of the DPP-4 inhibitor of this invention may be prepared according to known techniques.

Examples of suitable diluents for compounds according to embodiment A include cellulose powder, calcium hydrogen phosphate, erythritol, low substituted hydroxypropyl cellulose, mannitol, pregelatinized starch or xylitol.

Examples of suitable lubricants for compounds according to embodiment A include talc, polyethyleneglycol, calcium behenate, calcium stearate, hydrogenated castor oil or magnesium stearate.

Examples of suitable binders for compounds according to embodiment A include copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, or low-substituted hydroxypropylcellulose (L-HPC).

Examples of suitable disintegrants for compounds according to embodiment A include corn starch or crospovidone.

Suitable methods of preparing (oral) preparations or dosage forms of the DPP-4 inhibitors according to embodiment A of the invention are
direct tabletting of the active substance in powder mixtures with suitable tabletting excipients;
granulation with suitable excipients and subsequent mixing with suitable excipients and subsequent tabletting as well as film coating; or
packing of powder mixtures or granules into capsules.

Suitable granulation methods are
wet granulation in the intensive mixer followed by fluidised bed drying;
one-pot granulation;
fluidised bed granulation; or
dry granulation (e.g. by roller compaction) with suitable excipients and subsequent tabletting or packing into capsules.

An exemplary composition (e.g. tablet core) of a DPP-4 inhibitor according to embodiment A of the invention comprises the first diluent mannitol, pregelatinized starch as a second diluent with additional binder properties, the binder copovidone, the disintegrant corn starch, and magnesium stearate as lubricant; wherein copovidone and/or corn starch may be optional.

A tablet of a DPP-4 inhibitor according to embodiment A of the invention may be film coated, preferably the film coat comprises hydroxypropylmethylcellulose (HPMC), polyethylene glycol (PEG), talc, titanium dioxide and iron oxide (e.g. red and/or yellow).

In a further embodiment, the DPP-4 inhibitor according to the invention is preferably administered by injection (preferably subcutaneously). In another embodiment, the GLP-1 (GLP-1 mimetic or native GLP-1) having a short half life is preferably administered by injection (preferably subcutaneously) as well.

Injectable formulations of the GLP-1 (GLP-1 mimetic or native GLP-1) having a short half life and/or the DPP-4 inhibitor of this invention (particularly for subcutaneous use) may be prepared according to known formulation techniques, e.g. using suitable liquid carriers, which usually comprise sterile water, and, optionally, further additives such as e.g. preservatives, pH adjusting agents, buffering agents, isotoning agents, solubility aids and/or tensides or the like, to obtain injectable solutions or suspensions. In addition, injectable formulations may comprise further additives, for example salts, solubility modifying agents or precipitating agents which retard release of the drug(s). In further addition, injectable GLP-1 formulations may comprise GLP-1 stabilizing agents.

For example, an injectable formulation (particularly for subcutaneous use) containing the short-acting GLP-1 receptor agonist (e.g. exenatide), optionally together with the DPP-4 inhibitor of this invention, may further comprise the following additives: a tonicity-adjusting agent (such as e.g. mannitol), an antimicrobial preservative (such as e.g. metacresol), a buffer or pH adjusting agent (such as e.g. glacial acetic acid and sodium acetate trihydrate in water for injection as a buffering solution at pH 4.5), and optionally a solubilizing and/or stabilizing agent (such as e.g. a surfactant or detergent).

In a further embodiment, the DPP-4 inhibitor according to the invention is preferably administered by a transdermal delivery system. In another embodiment, the GLP-1 (GLP-1 mimetic or native GLP-1) having a short half life is preferably administered by a transdermal delivery system as well.

Transdermal formulations (e.g. for transdermal patches or gels) of the GLP-1 (GLP-1 mimetic or native GLP-1) having a short half life and/or the DPP-4 inhibitor of this invention may be prepared according to known formulation techniques, e.g. using suitable carriers and, optionally, further additives. To facilitate transdermal passage, different methodologies and systems may be used, such as e.g. techniques involving formation of microchannels or micropores in the skin, such as e.g. iontophoresis (based on low-level electrical current), sonophoresis (based on low-frequency ultrasound) or microneedling, or the use of drug-carrier agents (e.g. elastic or lipid vesicles such as transfersomes) or permeation enhancers.

The pharmaceutical compositions (or formulations) may be packaged in a variety of ways. Generally, an article for distribution includes one or more containers that contain the one or more pharmaceutical compositions in an appropriate form. Tablets are typically packed in an appropriate primary package for easy handling, distribution and storage and for assurance of proper stability of the composition at prolonged contact with the environment during storage. Primary containers for tablets may be bottles or blister packs.

A suitable bottle, e.g. for a pharmaceutical composition or combination (tablet) comprising a DPP-4 inhibitor according to embodiment A of the invention, may be made from glass or polymer (preferably polypropylene (PP) or high density polyethylene (HD-PE)) and sealed with a screw cap. The screw cap may be provided with a child resistant safety closure (e.g. press-and-twist closure) for preventing or hampering access to the contents by children. If required (e.g. in regions with high humidity), by the additional use of a desiccant (such as e.g. bentonite clay, molecular sieves, or, preferably, silica gel) the shelf life of the packaged composition can be prolonged.

A suitable blister pack, e.g. for a pharmaceutical composition or combination (tablet) comprising a DPP-4 inhibitor according to embodiment A of the invention, comprises or is formed of a top foil (which is breachable by the tablets) and a bottom part (which contains pockets for the tablets). The top foil may contain a metallic foil, particularly aluminium or aluminium alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as e.g. poly(vinyl chloride) (PVC) coated with poly(vinylidene chloride) (PVDC); or a PVC foil laminated with poly(chlorotrifluoroethylene) (PCTFE)) or a multi-layer polymer-metal-polymer foil (such as e.g. a cold-formable laminated PVC/aluminium/polyamide composition).

To ensure a long storage period especially under hot and wet climate conditions an additional overwrap or pouch made of a multi-layer polymer-metal-polymer foil (e.g. a laminated polyethylene/aluminium/polyester composition) may be used for the blister packs. Supplementary desiccant (such as e.g. bentonite clay, molecular sieves, or, preferably, silica gel) in this pouch package may prolong the shelf life even more under such harsh conditions.

Solutions for injection may be available in typical suitable presentation forms such as vials, cartridges or prefilled (disposable) pens, which may be further packaged.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

With respect to the first embodiment (embodiment A), the dosage typically required of the DPP-4 inhibitors mentioned herein in embodiment A when administered intravenously is 0.1 mg to 10 mg, preferably 0.25 mg to 5 mg, and when administered orally is 0.5 mg to 100 mg, preferably 2.5 mg to 50 mg or 0.5 mg to 10 mg, more preferably 2.5 mg to 10 mg or 1 mg to 5 mg, in each case 1 to 4 times a day. Thus, e.g. the dosage of 1-[(4-methyl-quinazolin-2-yl)methyl]-3-methyl-7-(2-butyn-1-yl)-8-(3-(R)-amino-piperidin-1-yl)-xanthine when administered orally is 0.5 mg to 10 mg per patient per day, preferably 2.5 mg to 10 mg or 1 mg to 5 mg per patient per day.

For example, doses of linagliptin when administered subcutaneously or i.v. for human patients are in the range of 0.3-10 mg, preferably from 1 to 5 mg, particularly 2.5 mg, per patient per day.

In a further embodiment, for example, doses of linagliptin when administered subcutaneously for human subjects (such as e.g. in obese human patients or for treating obesity) are in the range of 0.1-30 mg, preferably from 1 to 10 mg, particularly 5 mg, per patient per day.

A dosage form prepared with a pharmaceutical composition comprising a DPP-4 inhibitor mentioned herein in embodiment A contain the active ingredient in a dosage range of 0.1-100 mg. Thus, e.g. particular oral dosage strengths of 1-[(4-methyl-quinazolin-2-yl)methyl]-3-methyl-7-(2-butyn-1-yl)-8-(3-(R)-amino-piperidin-1-yl)-xanthine are 0.5 mg, 1 mg, 2.5 mg, 5 mg and 10 mg.

A special embodiment of the DPP-4 inhibitors of this invention refers to those orally administered DPP-4 inhibitors which are therapeutically efficacious at low dose levels, e.g. at oral dose levels <100 mg or <70 mg per patient per day, preferably <50 mg, more preferably <30 mg or <20 mg, even more preferably from 1 mg to 10 mg, particularly from 1 mg to 5 mg (more particularly 5 mg), per patient per day (if required, divided into 1 to 4 single doses, particularly 1 or 2 single doses, which may be of the same size, preferentially, administered orally once- or twice daily (more preferentially once-daily), advantageously, administered at any time of day, with or without food. Thus, for example, the daily oral amount 5 mg BI 1356 can be given in an once daily dosing regimen (i.e. 5 mg BI 1356 once daily) or in a twice daily dosing regimen (i.e. 2.5 mg BI 1356 twice daily), at any time of day, with or without food.

The GLP-1 analogue or mimetic having a short half life or the native GLP-1 are typically administered by subcutaneous injection, such as e.g. in an amount of 1-30 μg, 1-20 μg or 5-μg, e.g. once, twice or thrice daily. An embodiment thereof refers to those short-acting GLP-1 analogues (or any short-acting GLP-1 receptor agonists in general) that are to be administered at least twice daily, such as e.g. exenatide.

For example, exenatide is typically administered twice daily by subcutaneous injection (e.g. formulated as Byetta, e.g. in doses of 5-30 μg, particularly 5-20 μg, preferably 5-10 μg, specific dosage strengths are 5 or 10 μg).

The dosage of the active ingredients in the combinations and compositions in accordance with the present invention may be varied, although the amount of the active ingredients shall be such that a suitable dosage form is obtained. Hence, the selected dosage and the selected dosage form shall depend on the desired therapeutic effect, the route of administration and the duration of the treatment. Dosage ranges for the combination may be from the maximal tolerated dose for the single agent to lower doses, e.g. to one tenth of the maximal tolerated dose.

A particularly preferred DPP-4 inhibitor to be emphasized within the meaning of this invention is 1-[(4-methyl-quinazolin-2-yl)methyl]-3-methyl-7-(2-butyn-1-yl)-8-(3-(R)-amino-piperidin-1-yl)-xanthine (also known as BI 1356 or linagliptin). BI 1356 exhibits high potency, 24 h duration of action, and a wide therapeutic window. In patients with type 2 diabetes receiving multiple oral doses of 1, 2.5, 5 or 10 mg of BI 1356 once daily for 12 days, BI 1356 shows favourable pharmacodynamic and pharmacokinetic profile (see e.g. Table 3 below) with rapid attainment of steady state (e.g. reaching steady state plasma levels (>90% of the pre-dose plasma concentration on Day 13) between second and fifth day of treatment in all dose groups), little accumulation (e.g. with a mean accumulation ratio R_{A,AUC}≦1.4 with doses above 1 mg) and preserving a long-lasting effect on DPP-4 inhibition (e.g. with almost complete (>90%) DPP-4 inhibition at the 5 mg and 10 mg dose levels, i.e. 92.3 and 97.3% inhibition at steady state, respectively, and >80% inhibition over a 24 h interval after drug intake), as well as significant decrease in 2 h postprandial blood glucose excursions by ≧80% (already on Day 1) in doses ≧2.5 mg, and with the cumulative amount of unchanged parent compound excreted in urine on Day 1 being below 1% of the administered dose and increasing to not more than about 3-6% on Day 12 (renal clearance CL_{R,ss} is from about 14 to about 70 mL/min for the administered oral doses, e.g. for the 5 mg dose renal clearance is about 70 ml/min). In people with type 2 diabetes BI 1356 shows a placebo-like safety and tolerability. With low doses of about ≧5 mg, BI 1356 acts as a true once-daily oral drug with a full 24 h duration of DPP-4 inhibition. At therapeutic oral dose levels, BI 1356 is mainly excreted via the liver and only to a minor extent (about <7% of the administered oral dose) via the kidney. BI 1356 is primarily excreted unchanged via the bile. The fraction of BI 1356 eliminated via the kidneys increases only very slightly over time and with increasing dose, so that there will likely be no need to modify the dose of BI 1356 based on the patients' renal function. The non-renal elimination of BI 1356 in combination with its low accumulation potential and broad safety margin may be of significant benefit in a patient population that has a high prevalence of renal insufficiency and diabetic nephropathy.

TABLE 3
Geometric mean (gMean) and geometric coefficient of variation (gCV) of
pharmacokinetic parameters of BI 1356 at steady state (Day 12)

| | 1 mg | 2.5 mg | 5 mg | 10 mg |
|---|---|---|---|---|
| Parameter | gMean (gCV) | gMean (gCV) | gMean (gCV) | gMean (gCV) |
| AUC₀₋₂₄ | 40.2 (39.7) | 85.3 (22.7) | 118 (16.0) | 161 (15.7) |
| [nmol · h/L] | | | | |
| AUC_{T,ss} | 81.7 (28.3) | 117 (16.3) | 158 (10.1) | 190 (17.4) |
| [nmol · h/L] | | | | |
| Cₘₐₓ [nmol/L] | 3.13 (43.2) | 5.25 (24.5) | 8.32 (42.4) | 9.69 (29.8) |
| C_{max,ss} | 4.53 (29.0) | 6.58 (23.0) | 11.1 (21.7) | 13.6 (29.6) |
| [nmol/L] | | | | |
| tₘₐₓ * [h] | 1.50 [1.00-3.00] | 2.00 [1.00-3.00] | 1.75 [0.92-6.02] | 2.00 [1.50-6.00] |
| t_{max,ss} * [h] | 1.48 [1.00-3.00] | 1.42 [1.00-3.00] | 1.53 [1.00-3.00] | 1.34 [0.50-3.00] |
| T_{1/2,ss} [h] | 121 (21.3) | 113 (10.2) | 131 (17.4) | 130 (11.7) |
| Accumulation | 23.9 (44.0) | 12.5 (18.2) | 11.4 (37.4) | 8.59 (81.2) |
| t_{1/2,} [h] | | | | |
| R_{A,Cmax} | 1.44 (25.6) | 1.25 (10.6) | 1.33 (30.0) | 1.40 (47.7) |
| R_{A,AUC} | 2.03 (30.7) | 1.37 (8.2) | 1.33 (15.0) | 1.18 (23.4) |
| fe₀₋₂₄ [%] | NC | 0.139 (51.2) | 0.453 (125) | 0.919 (115) |
| fe_{T,ss} [%] | 3.34 (38.3) | 3.06 (45.1) | 6.27 (42.2) | 3.22 (34.2) |
| CL_{R,ss} | 14.0 (24.2) | 23.1 (39.3) | 70 (35.0) | 59.5 (22.5) |
| [mL/min] | | | | |

*median and range [min-max]
NC not calculated as most values below lower limit of quantification

As different metabolic functional disorders often occur simultaneously, it is quite often indicated to combine a number of different active principles with one another. Thus, depending on the functional disorders diagnosed, improved treatment outcomes may be obtained if a DPP-4 inhibitor is combined with active substances customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The DPP-4 inhibitors mentioned above—besides their use in mono-therapy—may also be used in conjunction with other active substances, by means of which improved treatment results can be obtained. Such a combined treatment may be given as a free combination of the substances or in the form of a fixed combination, for example in a tablet or capsule. Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide; nateglinide; repaglinide; mitiglinide; thiazolidinediones such as rosiglitazone and pioglitazone; PPAR gamma modulators such as metaglidases; PPAR-gamma agonists such as e.g. rivoglitazone, mitoglitazone, INT-131 and balaglitazone; PPAR-gamma antagonists; PPAR-gamma/alpha modulators such as tesaglitazar, muraglitazar, aleglitazar, indeglitazar and KRP297; PPAR-gamma/alpha/delta modulators such as e.g. lobeglitazone; AMPK-activators such as AICAR; acetyl-CoA carboxylase (ACC1 and ACC2) inhibitors; diacylglycerol-acetyltransferase (DGAT) inhibitors; pancreatic beta cell GCRP agonists such as GPR119 agonists (SMT3-receptor-agonists), such as the GPR119 agonists 5-ethyl-2-{4-[4-(4-tetrazol-1-yl-phenoxymethyl)-thiazol-2-yl]-piperidin-1-yl}-pyrimidine or 5-[1-(3-isopropyl-[1,2,4]oxadiazol-5-yl)-piperidin-4-ylmethoxy]-2-(4-methanesulfonyl-phenyl)-pyridine; 11β-HSD-inhibitors; FGF19 agonists or analogues; alpha-glucosidase blockers such as acarbose, voglibose and miglitol; alpha2-antagonists; insulin and insulin analogues such as human insulin, insulin lispro, insulin glusilin, r-DNA-insulinaspart, NPH insulin, insulin detemir, insulin degludec, insulin tregopil, insulin zinc suspension and insulin glargin; Gastric inhibitory Peptide (GIP); amylin and amylin analogues (e.g. pramlintide or davalintide); GLP-1 and GLP-1 analogues such as Exendin-4, e.g. exenatide, exenatide LAR, liraglutide, taspoglutide, lixisenatide (AVE-0010), LY-2428757 (a PEGylated version of GLP-1), dulaglutide (LY-2189265), semaglutide or albiglutide; SGLT2-inhibitors such as e.g. dapagliflozin, sergliflozin (KGT-1251), atigliflozin, canagliflozin, ipragliflozin, luseogliflozin or tofogliflozin; inhibitors of protein tyrosine-phosphatase (e.g. trodusquemine); inhibitors of glucose-6-phosphatase; fructose-1,6-bisphosphatase modulators; glycogen phosphorylase modulators; glucagon receptor antagonists; phosphoenolpyruvatecarboxykinase (PEPCK) inhibitors; pyruvate dehydrogenasekinase (PDK) inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976) or of serine/threonine kinases; glucokinase/regulatory protein modulators incl. glucokinase activators; glycogen synthase kinase inhibitors; inhibitors of the SH2-domain-containing inositol 5-phosphatase type 2 (SHIP2); IKK inhibitors such as high-dose salicylate; JNK1 inhibitors; protein kinase C-theta inhibitors; beta 3 agonists such as ritobegron, YM 178, solabegron, talibegron, N-5984, GRC-1087, rafabegron, FMP825; aldosereductase inhibitors such as AS 3201, zenarestat, fidarestat, epalrestat, ranirestat, NZ-314, CP-744809, and CT-112; SGLT-1 or SGLT-2 inhibitors; KV 1.3 channel inhibitors; GPR40 modulators such as e.g. [(3S)-6-({2′,6′-dimethyl-4′-[3-(methylsulfonyl)propoxy]biphenyl-3-yl}methoxy)-2,3-dihydro-1-benzofuran-3-yl]acetic acid; SCD-1 inhibitors; CCR-2 antagonists; dopamine receptor agonists (bromocriptine mesylate [Cycloset]); 4-(3-(2,6-dimethylbenzyloxy)phenyl)-4-oxobutanoic acid; sirtuin stimulants; and other DPP IV inhibitors.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

For children 10 to 16 years of age, the recommended starting dose of metformin is 500 mg given once daily. If this dose fails to produce adequate results, the dose may be increased to 500 mg twice daily. Further increases may be made in increments of 500 mg weekly to a maximum daily dose of 2000 mg, given in divided doses (e.g. 2 or 3 divided doses). Metformin may be administered with food to decrease nausea.

A dosage of pioglitazone is usually of about 1-10 mg, 15 mg, 30 mg, or 45 mg once a day.

Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

A dual combination of glibenclamide/metformin is usually given in doses from 1.25/250 once daily to 10/1000 mg twice daily. (typical dosage strengths are 1.25/250, 2.5/500 and 5/500 mg).

A dual combination of glipizide/metformin is usually given in doses from 2.5/250 to 10/1000 mg twice daily (typical dosage strengths are 2.5/250, 2.5/500 and 5/500 mg).

A dual combination of glimepiride/metformin is usually given in doses from 1/250 to 4/1000 mg twice daily.

A dual combination of rosiglitazone/glimepiride is usually given in doses from 4/1 once or twice daily to 4/2 mg twice daily (typical dosage strengths are 4/1, 4/2, 4/4, 8/2 and 8/4 mg).

A dual combination of pioglitazone/glimepiride is usually given in doses from 30/2 to 30/4 mg once daily (typical dosage strengths are 30/4 and 45/4 mg).

A dual combination of rosiglitazone/metformin is usually given in doses from 1/500 to 4/1000 mg twice daily (typical dosage strengths are 1/500, 2/500, 4/500, 2/1000 and 4/1000 mg).

A dual combination of pioglitazone/metformin is usually given in doses from 15/500 once or twice daily to 15/850 mg thrice daily (typical dosage strengths are 15/500 and 15/850 mg).

The non-sulphonylurea insulin secretagogue nateglinide is usually given in doses from 60 to 120 mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

Acarbose is usually given in doses from 25 to 100 mg with meals. Miglitol is usually given in doses from 25 to 100 mg with meals.

Examples of combination partners that lower the lipid level in the blood are HMG-CoA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists such as e.g. {4-[(R)-2-ethoxy-3-(4-trifluoromethyl-phenoxy)-propylsulfanyl]-2-methyl-phenoxy}-acetic acid; inhibitors of acyl-coenzyme A:cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); and ApoB100 antisense RNA.

A dosage of atorvastatin is usually from 1 mg to 40 mg or 10 mg to 80 mg once a day.

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin II receptor blockers (ARBs) such as telmisartan, candesartan, valsartan, losartan, irbesartan, olmesartan, azilsartan and eprosartan.

A dosage of telmisartan is usually from 20 mg to 320 mg or 40 mg to 160 mg per day.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2 inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### EXAMPLES

### Linagliptin s.c. Dosing and its DPP-4 Inhibition in Plasma

Linagliptin subcutaneous (s.c.) dosing and DPP-4 inhibition in plasma can be comparable in efficacy and duration of action to oral dosing, which may make it suitable for use in fixed combination e.g. with a GLP-1 (GLP-1 mimetic or native GLP-1) having a short half life:

Male ZDF rats (n=5) have been treated with different concentrations of BI 1356 in a subcutaneous (s.c.) administration regimen (0.001 mg/kg, 0.01 mg/kg, 0.1 mg/kg and 1 mg/kg in 0.5 ml/kg NaCl solution) in comparison to 3 mg/kg p.o. (in 0.5% Natrosol, 5 ml/kg volume of application).

DPP-4 activity in EDTA plasma was detected 1, 3, 5, 7, 24, 31, 48, 72 h following drug administration (blood was taken by venous puncture under isofluran anesthesia from the vena sublingualis).

Doses of BI 1356 from 0.01 mg/kg (s.c. administered) on demonstrated significant inhibition of DPP-4 activity compared to control. The dose of 0.1 mg/kg and 1 mg/kg (s.c. administration) of BI 1356 had a persistent DPP-4 inhibition of more than 64% over 7 h. The 1 mg/kg s.c. dose was comparable in efficacy and duration of action to the 3 mg/kg oral dose.

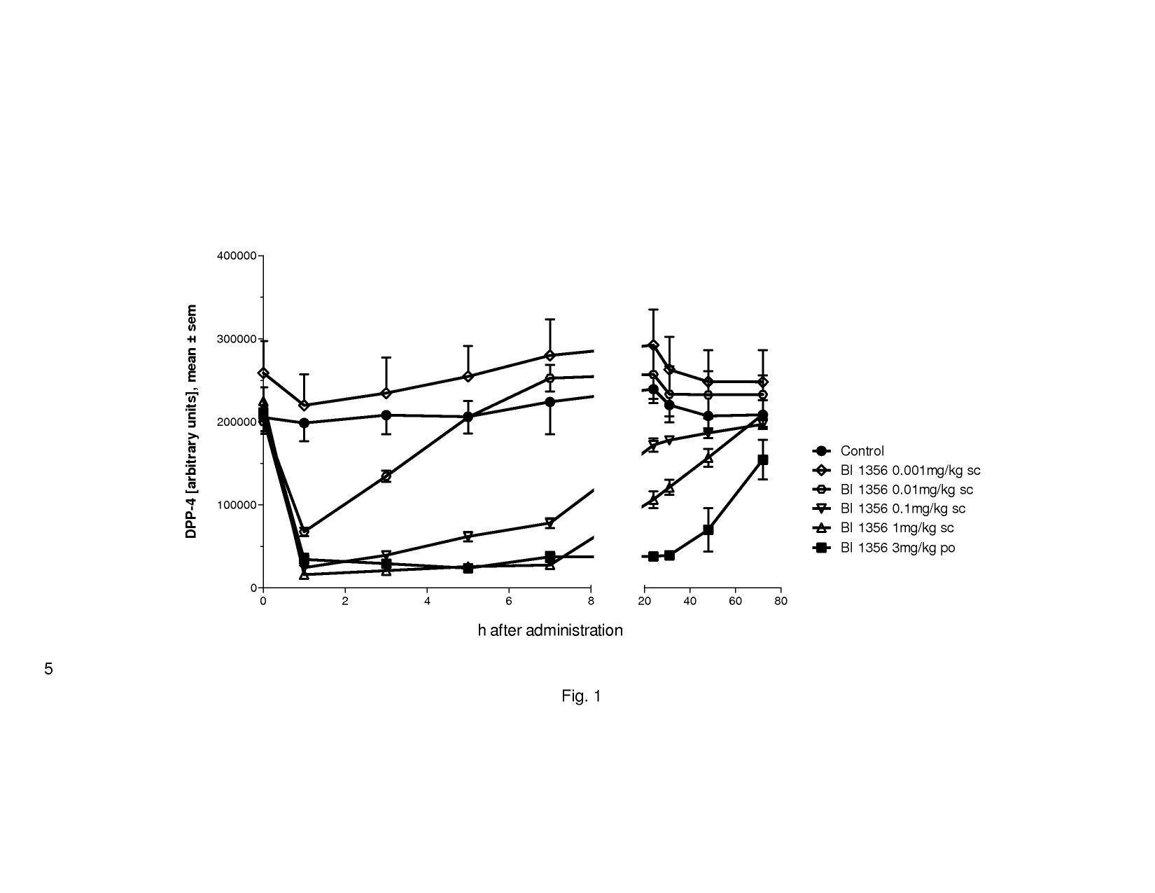

The results of the above studies are shown on FIG. 1

### Effect of Linagliptin on Body Weight Total Body Fat, Liver Fat and Intramyocellular Fat

In a further study the efficacy of chronic treatment with linagliptin on body weight, total body fat, intra-myocellular fat, and hepatic fat in a non-diabetic model of diet induced obesity (D10) in comparison to the appetite suppressant subutramine is investigated:

Rats are fed a high-fat diet for 3 months and received either vehicle, linagliptin (10 mg/kg), or sibutramine (5 mg/kg) for 6 additional weeks, while continuing the high-fat diet. Magnetic resonance spectroscopy (MRS) analysis of total body fat, muscle fat, and liver fat is performed before treatment and at the end of the study.

Sibutramine causes a significant reduction of body weight (−12%) versus control, whereas linagliptin has no significant effect (−3%). Total body fat is also significantly reduced by sibutramine (−12%), whereas linagliptin-treated animals show no significant reduction (−5%). However, linagliptin and sibutramine result both in a potent reduction of intramyocellular fat (−24% and −34%, respectively). In addition, treatment with linagliptin results in a profound decrease of hepatic fat (−39%), whereas the effect of sibutramine (−30%) does not reach significance (see Table 4). Thus, linagliptin is weight neutral but improves intra-myocellular and hepatic lipid accumulation.

TABLE 4
Effect of linagliptin on body weight total body fat, liver fat and intramyocellular fat

| | Body weight | | Total body fat | | Liver fat | | Intra-myocellular fat | |
|---|---|---|---|---|---|---|---|---|
| | % contr. | % baseli. | % contr. | % baseli. | % contr. | % baseli. | % contr. | % baseli. |
| Control | — | +15% | — | +11% | — | +27% | — | +23% |
| | | p = 0.016 | | p = 0.001 | | p = 0.09 | | p = 0.49 |
| Linagliptin | −3% | +12% | −5% | +5% | −39% | −30% | −36% | −24% |
| | p = 0.56 | p = 0.001 | p = 0.27 | p = 0.06 | p = 0.022 | p = 0.05 | p = 0.14 | p = 0.039 |
| Sibutramine | −12% | +1% | −12% | −0.4% | −30% | −29% | −55% | −34% |
| | p = 0.018 | p = 0.64 | p = 0.008 | p = 0.86 | p = 0.13 | p = 0.12 | p = 0.037 | p = 0.007 |

In conclusion, linagliptin treatment provokes a potent reduction of intramyocellular lipids and hepatic fat, which are both independent of weight loss. The treatment with linagliptin provides additional benefit to patients with diabetes who are additionally affected by liver steatosis (e.g. NAFLD). The effects of sibutramine on muscular and hepatic fat are attributed mainly to the known weight reduction induced by this compound.

### Delaying Onset of Diabetes and Preserving Beta-Cell Function in Non-Obese Type-1 Diabetes:

Though reduced pancreatic T-cell migration and altered cytokine production is considered important players for the onset of insulinitis the exact mechanism and effects on the pancreatic cell pool is still incompletely understood. In an attempt to evaluate the effect of linagliptin on pancreatic inflammation and beta-cell mass it is examined the progression of diabetes in the non-obese-diabetic (NOD) mice over a 60 day experimental period coupled with terminal stereological assessment of cellular pancreatic changes.

Sixty female NOD mice (10 weeks of age) are included in the study and fed a normal chow diet or a diet containing linagliptin (0.083 g linagliptin/kg chow; corresponding to 3-10 mg/kg, p.o) throughout the study period. Bi-weekly plasma samples are obtained to determine onset of diabetes (BG>11 mmol/l). At termination, the pancreata are removed and a terminal blood sample is obtained for assessment of active GLP-1 levels.

At the end of the study period the incidence of diabetes is significantly decreased in linagliptin-treated mice (9 out of 30 mice) compared with the control group (18 of 30 mice, p=0.021). The subsequent stereological assessment of beta-cell mass (identified by insulin immunoreactivity) demonstrates a significantly larger beta cell mass (vehicle 0.18±0.03 mg; linagliptin 0.48±0.09 mg, p<0.01) and total islet mass (vehicle 0.40±0.04 mg; linagliptin 0.70±0.09 mg, p<0.01) in linagliptin treated mice. There is a tendency for linagliptin to reduce peri-islet infiltrating lymphocytes (1.06±0.15; lina 0.79±0.12 mg, p=0.17). As expected active plasma GLP-1 are higher at termination in linagliptin treated mice. In summary, the data demonstrate that linagliptin is able to delay the onset of diabetes in a type-1 diabetic model (NOD mouse). The pronounced beta-cell sparing effects which can be observed in this animal model indicate that such DPP-4 inhibition not only protects beta-cells by increasing active GLP-1 levels, but may also exerts direct or indirect anti-inflammatory actions.

These effects may support the use of linagliptin in treating and/or preventing type 1 diabetes or latent autoimmune diabetes in adults (LADA). Linagliptin may offer a new therapeutic approach for patients with or at-risk of type 1 diabetes or LADA.

### Linagliptin Modulates Immune Pathogenesis in RIP-B7.1 Transgenic (Tg) Mice, an Experimental Model for Type 1 Diabetes:

Dipeptidyl peptidase (DPP)-4 inhibitors block incretin degradation by DPP-4. We assess whether the DPP-4 inhibitor linagliptin suppresses progression to hyperglycemia in an autoimmune diabetes mouse model (RIP-B7.1). As in humans, diabetes development in this model critically depends on activated CD8 T cells. Diabetes develops in RIP-B7.1 tg mice after a single intramuscular (i.m.) vaccination (vac) of proinsulin (PI) plasmid DNA. Linagliptin (3 mg/kg/day) or placebo are given orally for 1 wk before i.m. vac and continued for 6 wks.

Vac A: Diabetes is induced using a PI-encoding plasmid resulting in an aggressive insulitis. Vac B: vac with insulin A-chain encoding plasmid results in a delayed diabetes development compared to vac A. With vac A (n=20 tg mice), diabetes incidence is 80% 5 weeks after vac, whereas vac B (n=34) results in 79% incidence after 12 wks in placebotreated mice. Linagliptin does not stop the aggressive insulitic process (vac A; n=20) but significantly delays diabetes onset (80% incidence after wk 8 of follow-up [p<0.05] compared to 5 wks in placebo-treated mice). When a less aggressive insulitis is induced (vac B; n=16) linagliptin treatment again delays onset and preserved β-cell function since diabetes incidence does not exceed 62% during 14 wks follow up (control mice: [n=34] 92% incidence wk 14; p<0.05). FACS and ELISPOT show that islet antigen-specific CD8 T cells express high levels of IFN-γ with equal number in placebo- and linagliptin-treated mice. In the linagliptin-treated group, islet insulin content is partially preserved after diabetes onset. Serum levels of the regulatory cytokine IL-10 are significantly upregulated in linagliptintreated mice.

These data suggest that DPP-4 inhibition can modulate T cell-mediated immune pathogenesis. Since linagliptin has no impact on the number of IFN-γ producing T cells, it is suggested that DPP-4 inhibition predominantly alleviates cytokine-induced β-cell death.

Combined s.c. Administration of the DPP-IV Inhibitor Linagliptin and Native GLP-1 Induce Body Weight Loss and Appetite Suppression in DIO Rats, a Model of Obesity

Background and aims: Linagliptin is a dipeptidyl peptidase (DPP)-IV inhibitor approved for the treatment of type 2 diabetes. DPP-IV inhibitors are weight-neutral, suggesting that elevation of endogenous incretin levels is not sufficient to promote weight loss per se. However, it is not known whether DPP-IV inhibition in conjunction with glucagon-like peptide (GLP)-1 administration would influence body weight. We therefore evaluated the effect of the combination of linagliptin and native GLP-1 (7-36) administration on body weight in both normal-weight and diet-induced obese (D10) rats fed a sugar- and fat-rich diet for 12 weeks, and compared the effect with the GLP-1 analogue liraglutide alone.

Materials and methods: Normal-weight and DIO male Sprague-Dawley rats were used for acute and chronic dosing experiments, respectively. All rats were stratified to treatment groups according to individual body weight and whole-body fat mass. Linagliptin+GLP-1 combination treatment was evaluated in both acute and chronic treatment settings and compared with monotherapy and vehicle controls. In linagliptin+GLP-1 chronic dose experiments, DIO rats were initially subjected to linagliptin for 14 days, and then GLP-1 was added to linagliptin for a further 14 days. For comparison, DIO rats were exposed to 28 days of liraglutide monotherapy. All drugs were administered twice daily subcutaneously (s.c.). Results: Acute linagliptin (0.1-0.5 mg/kg) had no effect on nocturnal food intake in normal-weight rats, whereas GLP-1 (0.2-0.4 mg/kg) administration evoked a rapid-onset suppression of food intake; however, its effects were modest and short-lived. Interestingly, acute linagliptin+GLP-1 combination and liraglutide (0.2 mg/kg) mono treatment induced a robust hypophagic response lasting for 3 h and 18 h, respectively. Although 14 days of treatment revealed no effect with linagliptin or GLP-1 monotherapy in the DIO rat, continuation with linagliptin (0.5 mg/kg)+GLP-1 (0.4 mg/kg) combination for an additional 14 days induced a sustained decrease in body weight (−6.4±0.8%) and high-fat/high-carbohydrate food intake (−62±6.0%) with a significant increase in chow preference. In comparison, chronic liraglutide (0.2 mg/kg) treatment evoked a long-lasting hypophagic response with a weight loss of −10.8±0.5% and 12.2±0.6% at day 14 and 28, respectively. The anti-obesity effects of linagliptin+GLP-1 combination and liraglutide monotherapy were associated with a marked reduction of abdominal fat deposits.

Conclusion: These data demonstrate that combined treatment with linagliptin and GLP-1 synergistically reduces body weight and fat deposits in DIO rats, an effect which is associated with appetite suppression. Linagliptin and GLP-1 co-administration (e.g. each being administered s.c.) may therefore hold promise as a novel therapeutic principle for combined weight and diabetes management in obese patients.

These effects may support the use of linagliptin and GLP-1 co-administration (particularly each being administered s.c.) in a method of treating overweight or obesity, reducing body weight and/or body fat and/or suppressing appetite, especially in obese, overweight and/or diabetic patients (e.g. type 1 diabetes, type 2 diabetes or LADA patients, especially type 2 diabetes patients, being obese or overweight).

What is claimed is:

1) A pharmaceutical combination, composition or kit comprising linagliptin, and a GLP-1 analogue having a short half life or native GLP-1.

2) The combination, composition or kit according to claim 1, which is for subcutaneous administration of the active components.

3) The combination, composition or kit according to claim 1, which is for simultaneous administration of the active components.

4) The combination according to claim 1, wherein the GLP-1 analogue or native GLP-1 and the linagliptin are present each in separate dosage forms.

5) The combination according to claim 1, wherein the GLP-1 analogue or native GLP-1 and the linagliptin are present in the same dosage form.

6) The combination according to claim 1, wherein the GLP-1 analogue or native GLP-1 and the linagliptin are comprised in a pharmaceutical composition for subcutaneous injection administration.

7) The combination according to claim 1, wherein the GLP-1 analogue or native GLP-1 and the linagliptin are comprised in a pharmaceutical kit, where each of the active components is for subcutaneous injection administration.

8) A method of using the combination, composition or kit according to claim 1 for treating obesity or overweight, or reducing body weight or body fat, or suppressing appetite in a subject, said method comprising administering linagliptin subcutaneously and the GLP-1 analogue or native GLP-1 subcutaneously to the subject.

9) A method for treating and/or preventing obesity or overweight or for reducing body weight in a subject, said method comprising administering subcutaneously an effective amount of linagliptin and a GLP-1 analogue having a short half life or native GLP-1, to the subject.

10) The method according to claim 9, wherein the linagliptin and the GLP-1 analogue or native GLP-1 are administered simultaneously.

11) The method according to claim 9, wherein the linagliptin and the GLP-1 analogue or native GLP-1 are administered in the same subcutaneous pharmaceutical composition.

12) The method according to claim 9, wherein linagliptin is administered subcutaneously in an amount of from 0.1 to 30 mg or from 0.3 to 10 mg each per subject.

13) The method according to claim 9, wherein the subject is non-diabetic.

14) The method according to claim 9, wherein the subject has type 2 diabetes, type 1 diabetes or latent autoimmune diabetes (LADA).

15) The method according to claim 9, wherein linagliptin and the GLP-1 analogue or native GLP-1 are administered twice daily, simultaneously, and each subcutaneously.

16) A method of using linagliptin subcutaneously, wherein said method is selected from:
preventing, slowing the progression of, delaying or treating a metabolic disorder or disease selected from type 1 diabetes mellitus, type 2 diabetes mellitus, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, postprandial lipemia, hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome;
improving and/or maintaining glycemic control and/or for reducing of fasting plasma glucose, of postprandial plasma glucose, of postabsorptive plasma glucose and/or of glycosylated hemoglobin HbA1c;
preventing, slowing, delaying or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying or treating of complications of diabetes mellitus such as micro- and macrovascular diseases, such as nephropathy, micro- or macroalbuminuria, proteinuria, retinopathy, cataracts, neuropathy, learning or memory impairment, neurodegenerative or cognitive disorders, cardio- or cerebrovascular diseases, tissue ischaemia, diabetic foot or ulcus, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke;
reducing body weight and/or body fat and/or liver fat and/or intra-myocellular fat or preventing an increase in body weight and/or body fat and/or liver fat and/or intra-myocellular fat or facilitating a reduction in body weight and/or body fat and/or liver fat and/or intra-myocellular fat;
preventing, slowing, delaying or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing, delaying or treating non alcoholic fatty liver disease (NAFLD) including hepatic steatosis, non-alcoholic steatohepatitis (NASH) and/or liver fibrosis;
preventing, slowing the progression of, delaying or treating type 2 diabetes with failure to conventional antidiabetic mono- or combination therapy;
achieving a reduction in the dose of conventional antidiabetic medication required for adequate therapeutic effect;
reducing the risk for adverse effects associated with conventional antidiabetic medication; and/or
maintaining and/or improving the insulin sensitivity and/or for treating or preventing hyperinsulinemia and/or insulin resistance;
said method comprising administering subcutaneously a therapeutically effective amount of linagliptin, optionally in combination with one or more other therapeutic agents, to the patient in need thereof.

17) The method according to claim 16, wherein the amount of linagliptin subcutaneously administered is from 0.1 to 30 mg or from 0.3 to 10 mg, each per subject.

18) The method according to claim 16, wherein the amount of linagliptin subcutaneously to be administered is from 1 to 10 mg per subject, wherein said subject is an obese human patient.

19) The pharmaceutical combination, composition or kit according to claim 1, wherein the GLP-1 analogue having a short half life is exendin (exendin-4 or exenatide).

20) The method according to claim 8, wherein the subject has obesity, type 1 or type 2 diabetes or LADA.

21) The method according to claim 20, wherein the subject is obese or overweight.

22) The method according to claim 16, wherein the amount of linagliptin subcutaneously administered is 2.5 mg per day or 5 mg per day, each per human subject.
